(19) United States
(12) Patent Application Publication
(10) Pub. No.: US 2003/0082675 A1
(43) Pub. Date: May 1, 2003

(54) TRANSKARYOTIC DELIVERY OF THERAPEUTIC PRODUCTS

(76) Inventors: DOUGLAS A. TRECO, ARLINGTON, MA (US); MICHAEL W. HEARTLEIN, BOXBOROUGH, MA (US); BRIAN M. HAUGE, BEVERLY, MA (US); RICHARD F. SELDON, WELLESLEY, MA (US)

Correspondence Address: FISH & RICHARDSON PC, 225 FRANKLIN ST, BOSTON, MA, 02110, US

(21) Appl. No.: 09/225,718

(22) Filed: Jan. 6, 1999

Related U.S. Application Data
(63) Continuation of application No. 09/012,364, filed on Jan. 23, 1998, now abandoned.
Application No. 09/012,364 is a division of application No. 08/406,030, filed on Mar. 17, 1995, now Pat. No. 6,270,989.
Application No. 08/406,030 is a continuation-in-part of application No. 08/243,391, filed on May 13, 1994, now Pat. No. 5,641,670.
Application No. 08/243,391 is a continuation-in-part of application No. 07/985,586, filed on Dec. 3, 1992, now abandoned.
Application No. 08/243,391 is a continuation-in-part of application No. 07/911,533, filed on Jul. 10, 1992, now abandoned.
Application No. 08/243,391 is a continuation-in-part of application No. 07/787,840, filed on Nov. 5, 1991, now abandoned.
Application No. 08/243,391 is a continuation-in-part of application No. 07/789,188, filed on Nov. 5, 1991, now abandoned.

(51) Int. Cl. C12P 21/02; C12N 1/21; C07H 21/04; C12N 9/22; C12N 15/74

(52) U.S. Cl. 435/69.1; 435/199; 435/472; 435/320.1; 435/252.3; 536/23.2

(57) ABSTRACT

The invention relates to novel human DNA sequences, targeting constructs, and methods for producing novel genes encoding thrombopoietin, DNase I, and β-interferon by homologous recombination. The targeting constructs comprise at least: a) a targeting sequence; b) a regulatory sequence; c) an exon; and d) a splice-donor site. The targeting constructs, which can undergo homologous recombination with endogenous cellular sequences to generate a novel gene, are introduced into cells to produce homologously recombinant cells. The homologously recombinant cells are then maintained under conditions which will permit transcription of the novel gene and translation of the mRNA produced, resulting in production of either thrombopoietin, DNase I, or β-interferon. The invention further relates to a methods of producing pharmaceutically useful preparations containing thrombopoietin, DNase I, or β-interferon from homologously recombinant cells and methods of gene therapy comprising administering homologously recombinant cells producing thrombopoietin, DNase I, or β-interferon to a patient for therapeutic purposes.

### RELATED APPLICATIONS

This application is a Continuation-In-Part of U.S. patent application Ser. No. 08/243,391, filed May 13, 1994, which is a Continuation-In-Part of U.S. patent application Ser. No. 07/985,586, filed Dec. 3, 1992, and is also a Continuation-In-Part of U.S. patent application Ser. No. 07/911,533, filed Jul. 10, 1992, and is also a Continuation-In-Part of U.S. patent application Ser. No. 07/787,840, filed Nov. 5, 1991, and is also a Continuation-In-Part of U.S. patent application Ser. No. 07/789,188, filed Nov. 5, 1991, all of which are incorporated herein by reference. This application also claims priority and is related to PCT/US93/11704, filed Dec. 2, 1993, and is also related to PCT/US92/09627, filed Nov. 5, 1992. The teachings of PCT/US93/11704 and PCT/US92/09627 are incorporated herein by reference.

### BACKGROUND OF THE INVENTION

Current approaches to treating disease by administering therapeutic proteins include in vitro production of therapeutic proteins for conventional pharmaceutical delivery (e.g. intravenous, subcutaneous, or intramuscular Injection, or by intranasal or intratracheal aerosol administration) and, more recently, gene therapy.

One protein which may be useful in the treatment of platelet disorders is thrombopoietin (TPO). Platelets are small (2-3 microns in diameter) a nucleated cells which play an important role in primary hemostasis by adhering to and aggregating at sites of vascular damage. In addition, platelets release factors which are important components of the blood coagulation, inflammation, and wound healing pathways. Patients with very low levels of circulating platelets (thrombocytopenia) exhibit bleeding into superficial sites (e.g. skin, mucous membranes, genitourinary tract, and gastrointestinal tract) as a result of mild trauma, and are at risk for death from catastrophic hemorrhage occurring spontaneously or resulting from trauma. The physiologic role of platelets and the etiology of platelet disorders have been described (cf. Hematology: Clinical and Laboratory Practice, Eds. R. L. Bick et al., pp. 1337-1389, Mosby, St. Louis (1993); Harrison's Principles of Internal Medicine, Eds. J. D. Wilson et al., 11th Ed., pp. 1500-1505, McGraw Hill, New York, 1991).

Thrombocytopenia may be caused by decreased production of platelets by the bone marrow, increased sequestration of platelets in the spleen, or accelerated platelet destruction. Decreased production of platelets by the bone marrow may result from destruction of hematopoietic precursor cells by irradiation or treatment with cytotoxic agents during therapy for cancer. In addition, alcohol, estrogens, and thiazide diuretics can suppress platelet production (drug-induced thrombocytopenia). Furthermore, infiltration of the bone marrow by malignant cells and the disorders congenital amegakaryocytic hypoplasia and thrombocytopenia with absent radii (TAR syndrome) can result in decreased platelet production.

Increased splenic sequestration of platelets may occur as a result from splenomegaly associated with a variety of conditions, including liver disease, infiltration of the spleen with tumor cells as in myeloproliferative or lymphoproliferative disorders, and Gaucher's disease.

Accelerated platelet destruction and thrombocytopenia may be caused by vasculitis, hemolytic uremic syndrome, disseminated intravascular coagulation, and the presence of intravascular prosthetic devices such as cardiac valves. In addition, certain viral infections, drugs, and autoimmune disorders lead to immunologic thrombocytopenia in which platelets become coated with antibody, immune complexes, or complement and are rapidly cleared from the circulation. A number of drugs can elicit an immune response leading to immunologic thrombocytopenia, including sulfathiazole, novobiocin, para-aminosalicylate, quinidine, quinine, carbamazepine, digitoxin, arsenical drugs, and methyldopa.

Thrombocytopenia is currently treated most readily by transfusion with platelet concentrates, although corticosteroid therapy or plasmapheresis can be effective in immunologic thrombocytopenia. Treatment with platelet concentrates is severely limited by availability of suitable donors and the risk of transmission of blood-borne infectious diseases.

As an alternative to transfusion therapy, platelet deficiencies could be treated with hematopoietic growth factors which promote proliferation and maturation of megakaryocytes, the nucleated progenitor cells from which platelets are derived. Recently, cDNA clones were isolated which encode the human, mouse, and dog analogs of a protein purified from aplastic porcine plasma which displays megakaryocytopoietic activity (de Sauvage, F. J. et al. Nature 369:533-538 (1994); Lok, S. et al. Nature 369:565-568 (1994); Bartley, T. D. et al. Cell 77:1117-1124 (1994)). The encoded protein, termed thrombopoietin (TPO), stimulates proliferation and maturation of megakaryocytes and induces platelet production in vivo upon injection into experimental animals.

Methods for the production and delivery of other proteins with therapeutic properties are desirable. For example, it has been demonstrated that recombinant β-interferon is an effective medication for treatment of exacerbations in patients with relapsing-remitting multiple sclerosis (MS; see Kelley, C. L. and Smeltzer, S. C. J. Neuroscience Nursing 26:52-56 (1994)). Furthermore, it has been reported that β-interferon isolated from non-transfected cultured human fibroblasts may be an effective means for preventing the progression of acute non-A, non-B hepatitis to chronic disease (Omata, M. et al., Lancet 338:914-915 (1991)).

As another example, it has been demonstrated that recombinant human DNase I is an effective agent for reducing the viscosity of sputum from cystic fibrosis (CF) patients (Shak, S. et al., Proc. Natl. Acad. Sci. USA 87:9188-9192 (1990)) and for improving pulmonary function and decreasing exacerbations of respiratory disease in CF patients (Fuchs, H. J. et al., New Engl. J. Med. 331:637-642 (1994)). It has been further suggested that DNase I may be effective in improving respiratory function in patients with other respiratory diseases, such as chronic bronchitis and pneumonia (Shak, S. et al. , op. cit.).

While TPO, β-interferon, and DNase I are useful, for example, in the treatment of thrombocytopenia, MS, and CF, respectively, production of therapeutic proteins using genetic engineering technology as taught in the prior art is limited to conventional recombinant DNA methods, in which the recombinant protein is purified from mammalian cells expressing an exogenous cloned gene or cDNA under the control of a suitable promoter. The exogenous DNA encoding the protein of interest is introduced into cells in the form of a viral vector, circular plasmid DNA, or linear DNA fragment. Chinese Hamster Ovary (CHO) cell lines and their derivatives (Gottesman, M. M. Meth. Enzymol. 151:3-8 (1987) or mouse cell lines, such as NSO (Galfre, G. and Milstein, C., Meth. Enzymol. 73(B): 3-46 (1981)) or P3X63Ag8.653 (Kearney, J. et al. J. Immunol. 123: 1548-1550 (1979)) are commonly used, and the production of human therapeutic proteins is thus accomplished by expression and purification of the protein from a cell of non-human origin.

In many cases, it is desirable to produce human therapeutic proteins in a human cell, for example, when it is desired that the glycosylation pattern of the protein be similar to patterns normally found on human cells. In addition, the expression of human proteins in human cells is important in the development of gene therapy methods, in which a patient's cells are engineered to produce a desired therapeutic protein to alleviate the symptoms or cure a disease.

Clearly, the development of novel methods for the production of these human proteins in human cells would be of benefit to patients, through the availability of a wider range of products with therapeutic effectiveness. One approach proposed by scientists in the field for accomplishing this goal is to use homologous recombination, or gene targeting, to introduce a cloned, exogenous regulatory element (i.e. a promoter and/or enhancer) into a cell's genome at a pre-selected site such that the regulatory element activates expression of a nearby gene, ultimately resulting in production of the protein encoded by that gene. This approach has been suggested in U.S. Pat. No. 5,272,071 and in foreign patent applications WO 91/06666, WO 91/06667 and WO 90/11354.

### SUMMARY OF THE INVENTION

Described herein are new methods for producing TPO, DNase I, and β-interferon through the generation of novel transcription units within a cell's genome, methods which differ dramatically from those in the art and represent a major advance in the ability to manipulate expression in mammalian cells. The methods are based on the fact that an exogenous regulatory sequence, an exogenous exon, either coding or non-coding, and a splice-donor site can be introduced into a preselected site in the genome by homologous recombination. The resulting cells are referred to as targeted or homologously recombinant cells. The introduced DNA is positioned such that transcripts under the control of the exogenous regulatory region include both the exogenous exon and endogenous exons present in either the TPO, DNAse I, or β-interferon genes, resulting in transcripts in which the exogenous and endogenous exons are operatively linked. The novel transcription units produced by homologous recombination allow TPO, DNAse I, or β-interferon to be produced in human cells using the naturally-occurring endogenous exons encoding these proteins without introducing any portion of the coding sequences of the cognate genes. The present invention further relates to improved materials and methods for both the in vitro production of TPO, β-interferon, and DNase I and for the production and delivery of TPO, β-interferon, and DNase I by gene therapy.

The methods of the present invention teach the production of TPO, β-interferon, or DNase I by gene activation, in which the coding DNA sequence of the corresponding protein is not introduced into a cell by transfection of exogenous DNA encoding the protein. Instead, noncoding sequences upstream of one of these genes or coding or noncoding sequences within the genes are manipulated by gene targeting to create a novel transcription unit which expresses TPO, β-interferon, or DNase I. It is a purpose of this invention to define sequences upstream of the TPO, β-interferon, or DNase I genes, non-coding sequences (introns and 5′ non-translated sequences) within the human TPO, β-interferon, or DNase I genes, and methods for utilizing these sequences for the production of TPO, β-interferon, or DNase I.

The methods described herein teach production of TPO, β-interferon, or DNase I proteins, by the generation of novel genes in which exogenous and endogenous exons are operatively linked. As a result of introduction of exogenous components into the chromosomal DNA of a cell, the expression of the protein encoded by the endogenous gene is activated. Other forms of altered gene expression may be envisioned, such as increasing expression of a gene which is expressed in the cell as obtained, changing the pattern of regulation or induction such that it is different than occurs in the cell as obtained, and reducing (including eliminating) expression of a gene which is expressed in the cell as obtained. For example, it may be desirable to perform in vitro protein production or gene therapy to produce a protein other than TPO, DNase I, or β-interferon using a cell type that naturally produces one of these proteins. In these settings, it would be desirable to eliminate expression of TPO, DNase I, or β-interferon.

The present invention further relates to DNA constructs useful in the method of activation of the TPO, β-interferon, or DNase I genes. The DNA constructs comprise: (a) targeting sequences; (b) a regulatory sequence; (c) an exon; and (d) an unpaired splice-donor site. The targeting sequence in the DNA construct is derived from chromosomal DNA lying within and/or upstream of the desired gene and directs the integration of elements (a)-(d) into the chromosomal DNA in a cell such that the elements (b)-(d) are operatively linked to sequences of the desired endogenous gene. In another embodiment, the DNA constructs comprise: (a) a targeting sequence, (b) a regulatory sequence, (c) an exon, (d) a splice-donor site, (e) an intron, and (f) a splice-acceptor site, wherein the targeting sequence in the DNA construct is derived from chromosomal DNA lying within and/or upstream of the desired gene and directs the integration of elements (a)-(f) such that the elements of (b)-(f) are operatively linked to the desired endogenous gene. The targeting sequence is homologous to the preselected site within or upstream of the TPO, β-interferon, or DNase I genes in the cellular chromosomal DNA with which homologous recombination is to occur. In the construct, the exon is generally 3′ of the regulatory sequence and the splice-donor site is 3′ of the exon. Constructs of this type are disclosed in pending U.S. patent applications U.S. Ser. No. 07/985,586 and U.S. Ser. No. 08/243,391, all of which are incorporated herein by reference.

The following serves to illustrate two embodiments of the present invention, in which the sequences upstream of the TPO gene are altered to allow expression of TPO in primary, secondary, or immortalized cells which do not express TPO in detectable quantities in their untransfected state as obtained. In embodiment 1 (FIG. 1), the targeting construct contains two targeting sequences. Both the first and second targeting sequences are homologous to sequences upstream of the TPO coding region, with the first targeting sequence 5′ of the second targeting sequence. The targeting construct also contains a regulatory region, an exon (which in this case, comprises noncoding sequences and begins at a CAP site) and an unpaired splice-donor site. The homologous recombination event that generates the novel transcription unit producing TPO is shown in FIG. 1.

In embodiment 2 (FIG. 2), the targeting construct also contains two targeting sequences. The first targeting sequence is homologous to sequences upstream of the endogenous TPO coding region, and the second targeting sequence is homologous to the second intron of the TPO gene. The targeting construct also contains a regulatory region, an exon (in this case a coding exon derived from the human growth hormone (hGH) gene) and an unpaired splice-donor site. The homologous recombination event that generates the novel transcription unit producing TPO is shown in FIG. 2.

In these two embodiments, the products of the targeting events are novel transcription units which generate a mature mRNA in which an exogenous exon is positioned upstream of exon 2 (Embodiment 1) or exon 3 (Embodiment 2) of the endogenous TPO gene. The product of transcription, splicing, translation, and post-translational cleavage of the signal peptide is mature TPO. Embodiments 1 and 2 differ with respect to the relative positions of the regulatory sequences of the targeting construct that are inserted and the specific pattern of splicing that needs to occur to produce the final, processed transcript.

The invention further relates to a method of producing TPO, β-interferon, or DNase I in vitro or in vivo through introduction of a construct as described above into host cell chromosomal DNA by homologous recombination to produce a homologously recombinant cell. The homologously recombinant cell is then maintained under conditions which will permit transcription, translation and secretion of TPO, β-interferon, or DNase I.

The present invention also relates to cells, such as homologously recombinant primary or secondary cells (i.e., non-immortalized cells) and homologously recombinant immortalized cells, useful for producing TPO, β-interferon, or DNase I, methods of making such cells, methods of using the cells for in vitro protein production, and methods of gene therapy. Homologously recombinant cells of the present invention are of vertebrate origin, particularly of mammalian origin, and even more particularly of human origin. Homologously recombinant cells produced by the method of the present invention contain exogenous DNA which causes the homologously recombinant cells to express a desired gene at a higher level or with a pattern of regulation or induction that is different than occurs in the corresponding cell that has not undergone homologous recombination.

In one embodiment, the activated TPO, β-interferon, or DNase I gene can be further amplified by the inclusion of an amplifiable selectable marker gene which has the property that cells containing amplified copies of the selectable marker gene can be selected for by culturing the cells in the presence of the appropriate selectable agent. The activated gene is amplified in tandem with the amplifiable selectable marker gene. Cells containing many copies of the activated gene are useful for in vitro protein production and gene therapy.

Homologously recombinant cells of the present invention are useful in a number of applications in humans and animals. In one embodiment, the cells can be implanted into a human or an animal for protein delivery in the human or animal. For example, TPO, DNase I, or β-interferon can be delivered systemically or locally in humans for therapeutic benefit in the treatment of disease (TPO for thrombocytopenia, DNase I for CF, or β-interferon for the treatment of MS). In addition, homologously recombinant non-human cells producing TPO, DNase I, or β-interferon of non-human origin may be produced, and human or non-human cells expressing TPO, DNase I, or β-interferon may be enclosed within barrier devices and implanted into humans or animals for use in a therapy.

### BRIEF DESCRIPTION OF THE DRAWINGS

FIG. 1 is a schematic diagram of a strategy for transcriptionally activating the TPO gene by the creation of a novel transcription unit; thick lines: targeting sequences; thin lines: introns and 5′ upstream region; cross-hatched box, regulatory sequence; stippled boxes: noncoding exon sequences; black boxes: coding exon sequences; open boxes: splice sites. The splice-donor site (SD) of the exogenous exon in the targeting construct and the splice-acceptor site (SA) flanking TPO exon 2 which is involved in splicing to the exogenous exon are indicated.

FIG. 2 is a schematic diagram of a strategy for transcriptionally activating the TPO gene by the creation of a novel transcription unit; thick lines: targeting sequences; thin lines: intron 1 and 5′ upstream region; cross-hatched box: regulatory sequence; stippled boxes: noncoding exon sequences; black boxes: coding exon sequences; open boxes, splice sites. The splice-donor site (SD) of the exogenous exon in the targeting construct and the splice-acceptor site (SA) flanking TPO exon 3 which is involved in splicing to the exogenous exon are indicated.

FIG. 3 presents the 6,943 bp genomic XbaI fragment encompassing the 5′ flanking region and exons 1, 2, and 3 of the human thrombopoietin (TPO) gene. The XbaI fragment is depicted by the solid line, while exons 1, 2, and 3 are represented by the solid boxes. The nucleotide positions of the ApaI, BamHI, HindIII, EcoRI, NotI, SfiI and XbaI recognition sequences are indicated. Nucleotides are numbered starting at the hTPO ATG initiation codon.

FIGS. 4A-4D present the nucleotide sequence of 4,488 bp of genomic DNA (SEQ ID NO: 3) from the human TPO locus lying 5′ to the known cDNA sequence (de Sauvage et al., op. cit.). Nucleotide numbers are noted at the beginning of each line. Numbering is based on the ATG initiation codon at position 1 (see FIGS. 5A-5B). Ambiguities in the nucleotide sequence are represented using the following code: R=A or G (purine); H=A, C, or T; V=A, C, or G; N_=A, C, G, or T; K=G or T; S=G or C; W=A or T. The recognition sites for ApaI, BamHI, HindIII, NotI, SfiI and XbaI and their corresponding nucleotide positions are indicated above the sequence.

FIGS. 5A-5B present the nucleotide sequence of 2,455_bp of genomic DNA (SEQ ID NO: 4) from the human TPO locus extending downstream from the position of the 5′ end of the known cDNA sequence (de Sauvage et al., op. cit.). Nucleotide numbers are noted at the beginning of each line. Numbering is based on the ATG initiation codon at position_{—}1. Shown are exon 1, intron 1, exon 2, intron 2, exon 3, and a portion of intron 3. Exons 1, 2, and 3 are underlined, and the coding portions of exons 2 and 3 are noted as underlined triplets. The intron-exon boundaries are deduced from the published cDNA sequence (de Sauvage et al., op. cit.). The recognition sites for ApaI, ScoRI, and XbaI and their corresponding nucleotide positions are indicated above the sequence.

FIG. 6 is a schematic diagram of the strategy for activating the human TPO gene using targeting construct pTPO1 as described in Example 2. The positions of the dhfr and neo markers, the exogenous CMV promoter and TPO exons 1-3 are indicated. Thick lines: targeting sequences; thin lines: introns and 5′ upstream region; cross-hatched box: CMV promoter; stippled boxes: noncoding exon sequences; black boxes: coding exon sequences; open boxes, splice sites. The splice-donor site (SD) of the exogenous exon in the targeting construct and the splice-acceptor site (SA) flanking TPO exon 3 which is involved in splicing to the exogenous exon are indicated. Recognition sites for BamHI (B), NotI (N), ClaI (C), XhoI (X), and XbaI which are relevant to the construction of the targeting construct are marked.

FIG. 7 is a schematic diagram of the strategy for activating the human TPO gene using targeting construct pTPO2 as described in Example 2. The positions of the dhfr and neo markers, the exogenous CMV promoter and TPO exons 1-3 are indicated. Thick lines: targeting sequences; thin lines: introns and 5′ upstream region; cross-hatched box: CMV promoter; heavily stippled boxes: noncoding exons from the CMV IE gene; lightly stippled boxes: noncoding exon sequences of TPO exons 1 and 2; black boxes: coding exon sequences of TPO exons 2 and 3; open boxes: splice sites. The splice-donor (SD) and splice-acceptor (SA) sites flanking the noncoding exons in the targeting construct and the splice-acceptor site (SA) flanking TPO exon 2 which is involved in splicing to the unpaired splice-donor site of the 3′ exogenous exon are indicated. Recognition sites for BamHI (B), HindIII (H), NotI (N), ClaI (C), SalI (S), EcoRI (R), and XbaI which are relevant to the construction of the targeting construct are marked.

FIG. 8 is a schematic diagram of the strategy for activating the human TPO gene using targeting construct pTPO3 as described in Example 2. The positions of the dhfr and neo markers, the exogenous CMV promoter and TPO exons 1-3 are indicated. Thick lines: targeting sequences; thin lines: introns and 5′ upstream region; cross-hatched box: CMV promoter; stippled boxes: noncoding exon sequences of TPO exons 1 and 2; black boxes: coding exon sequences (the coding exon corresponding to hGH exon 1 in the targeting construct and in the novel transcription unit is marked); open boxes: splice sites. The splice-donor site (SD) of the exogenous exon in the targeting construct and the splice-acceptor site (SA) flanking TPO exon 3 which is involved in splicing to the exogenous exon are indicated. Recognition sites for BamHI (B), HindIII (H), ClaI (C), XhoI (X), EcoRI (R), and XbaI which are relevant to the construction of the targeting construct are marked.

FIG. 9 is a diagrammatic representation of the approximately 8 kb HincII fragment encompassing the 5′ flanking region, exons 1 and 2, and the sequences downstream of exon 2 of the human DNase I gene. The HincII fragment is depicted by the solid line, while exons 1 and 2 are represented by solid rectangular boxes. The nucleotide positions of the ApaI, BamHI, HincII, EspI, SphI and SmaI recognition sequences are indicated. Nucleotides are numbered starting at the AUG initiation codon. The nucleotide positions which reside upstream of exon 2 are based on the DNA sequence presented in FIGS. 10 and 11.

FIGS. 10A-10D present the nucleotide sequence encompassing 4,042 bp of DNA (SEQ ID NO: 17) from the human DNase I locus lying 5′ to the known cDNA sequence (Shak, S. et al. op. cit.). Nucleotides numbers are noted at the beginning of each line. Numbering is based on the ATG initiation codon at position 1 (see FIG. 11). The recognition sites, and the corresponding nucleotide positions for ApaI, BamHI, HincII, EspI, and SphI are indicated above the sequence.

FIG. 11 presents the nucleotide sequence of 810 bp of DNA (SEQ ID NO: 18) from the human DNase I locus extending downstream from the position of the 5′ end of the known cDNA sequence (Shak, S. et al. op. cit.). Shown are exon 1, intron 1, and a portion of exon 2. Exon 1 and 2 sequences are underlined and the coding sequences are noted as underlined triplets. The positions of the putative CAP site and the AUG initiation codon are indicated. The intron-exon boundaries are deduced from the published cDNA sequence (Shak S. et al., op. cit.).

FIG. 12 shows a strategy for activation of the human DNase I gene by homologous recombination. The targeting fragment is a 4633 bp BamHII fragment from pDNaseI which contains; 283 bp of 5′ targeting sequence from position

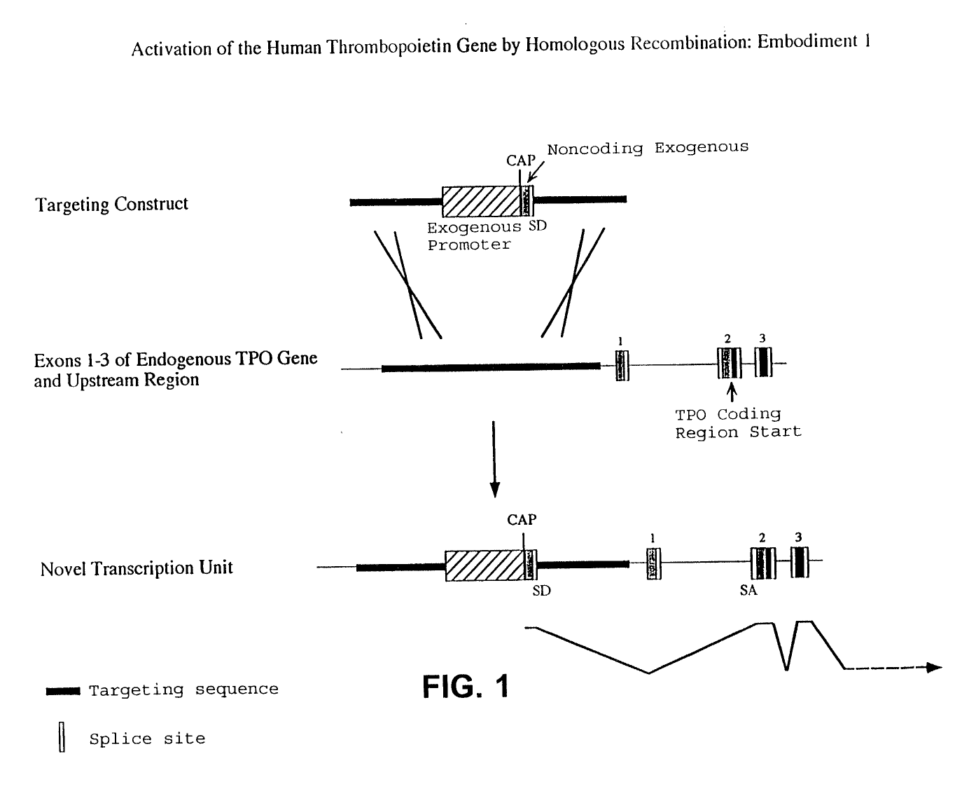

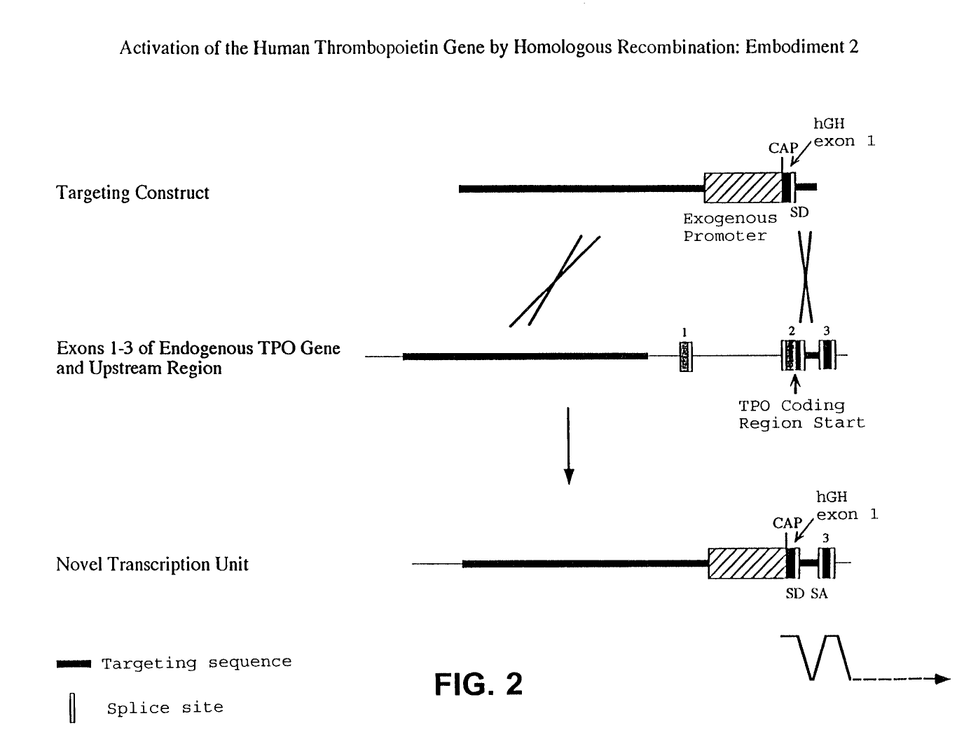

1162 (BamHII site) to −860 (ApaI site), an amplifiable dhfr expression unit, neo gene, CMV IE promoter, a CAP site, a non-codon exon, an unpaired splice-donor site and 363 bp of 3′ targeting sequence from position −860 (EspI site) to −468 (BamHI site). The dhfr expression unit and the neo gene are depicted by open arrows, the orientation of the arrows represent the direction of transcription. The positions of the CMV promoter, TATA box, CAP site and splice donor sequence (SD) are indicated. Activation of the DNase I gene is achieved by integration of the targeting fragment into the genome of the recipient cells by homologous recombination. The targeted gene product is depicted in the lower panel of the figure. The mRNA precursor which includes a non-coding 5′ exon, a chimeric intron and exon 2 of the DNase gene, is represented by the thin arrow.

FIG. 13 is a diagrammatic representation of 9,939 bp encompassing the 5′ flanking region, coding sequence and the 3′ untranslated region of the human β-interferon gene. The 5′ and 3′ flanking regions are depicted by the solid line and the transcribed region is represented by the solid box. The nucleotide positions of the BalI, BglII, EcoRI and PvuII recognition sequences are indicated. Nucleotides are numbered starting at the β-interferon ATG translational initiation codon (see FIG. 15).

FIGS. 14A-14G present the nucleotide sequence of 8,355 bp of DNA (SEQ ID NO: 23) from the human β-interferon locus lying 5′ to the known sequence (GenBank HUMIFNB1F). Nucleotide numbers are noted at the beginning of each line. Numbering is based on the ATG initiation codon at position 1 (see FIGS. 15). The recognition sites for BglII, EcoRI and PvuII and their corresponding nucleotide positions are indicated above the sequence.

FIGS. 15A-15B present the nucleotide sequence of 1,584 bp of DNA (SEQ ID NO: 24) from the human β-interferon locus extending downstream from the 5′ end of the known sequence (GenBank HUMIFNB1F). Nucleotide numbers are noted at the beginning of each line. Numbering is based on the ATG initiation codon at position 1. The transcribed region is underlined and the coding sequences are noted as underlined triplets. The position of the CAP site and AUG initiation codon are indicated. The recognition sites for BalI, BglII and PvuII and their corresponding nucleotide positions are indicated above the sequence.

FIG. 16 depicts the strategy for activation of the human β-interferon gene by homologous recombination using targeting construct pIFNb-1 as described in Example 7. The positions of the TATA box, CAP site, dhfr and neo markers, the exogenous CMV promoter, and the β-interferon 5′ flanking region and coding sequence are indicated. Thick lines: targeting sequences; thin lines: intron, β-interferon 5′ and 3′ non-coding sequences; solid box: CMV promoter; shaded box: endogenous β-interferon transcribed region; cross-hatched box: non-coding CMV exon 1 and the chimeric exon 2. The splice-donor site (SD) of the exogenous exon and the splice-acceptor site (SA) flanking the chimeric exon 2 are indicated. Recognition sites for BamHI, EcoRI, HincII, NdeI and PvuII which are relevant to the construction of the targeting construct are marked.

### DETAILED DESCRIPTION OF THE INVENTION

The present invention as set forth above, relates to a method of expressing TPO, DNase I, or β-interferon in human cells by activation of the endogenous TPO, DNase I, or β-interferon genes. In the present invention, homologous recombination is used to insert a regulatory region, an exon, and a splice-donor site upstream of endogenous exons coding for TPO, DNase I, or β-interferon, generating novel transcription units which are active in the homologously recombinant cell produced. The present invention further relates to homologously recombinant cells produced by the present method and to uses of the homologously recombinant cells. In a related embodiment, an activated TPO, DNase I, or β-interferon gene is amplified subsequent to activation, thus allowing enhanced expression of the activated gene.

The invention is based upon the discovery that the regulation or activity of endogenous genes of interest in a cell can be altered by creating a novel gene, in which the transcription product of the gene combines exogenous and endogenous exons and is under the control of an exogenous promoter. The method is practiced by inserting into a cell's genome, at a preselected site, through homologous recombination, DNA constructs comprising: (a) one or more targeting sequences; (b) a regulatory sequence; (c) an exon and (d) an unpaired splice-donor site, wherein the targeting sequence or sequences are derived from chromosomal DNA within and/or upstream of a desired endogenous gene and directs the integration of elements (a)-(d) such that the elements (b)-(d) are operatively linked to the endogenous gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence or sequences are derived from chromosomal DNA within and/or upstream of a desired endogenous gene and directs the integration of elements (a)-(f) such that the elements of (b)-(f) are operatively linked to the first exon of the endogenous gene.

The present invention relates particularly to novel DNA sequences that can be used in the construction of targeting constructs. Non-coding genomic DNA sequences within and upstream of the transcribed regions of the TPO and DNase I genes, and upstream of the transcribed region of the β-interferon gene, were cloned and are described for the first time. These sequences or DNA fragments comprising these sequences may be used as targeting sequences in DNA constructs useful for gene activation by homologous recombination. Typically, a targeting sequence is at least about 20 base pairs in length. The size of the sequence is chosen to be a size which selectively promotes homologous recombination with desired genomic DNA sequences.

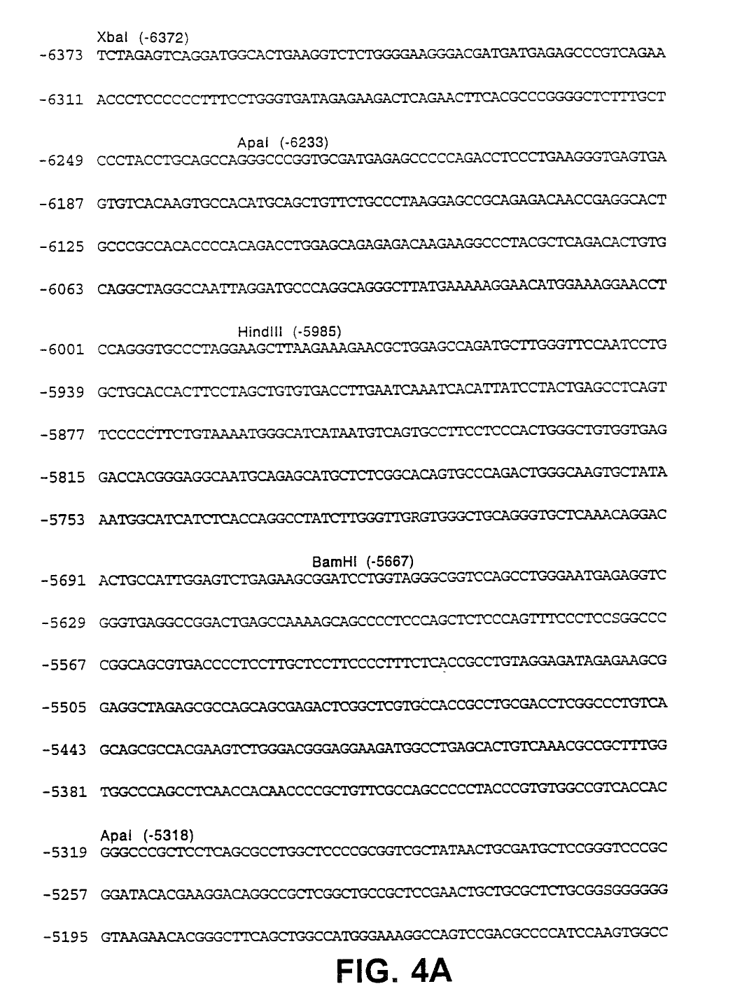

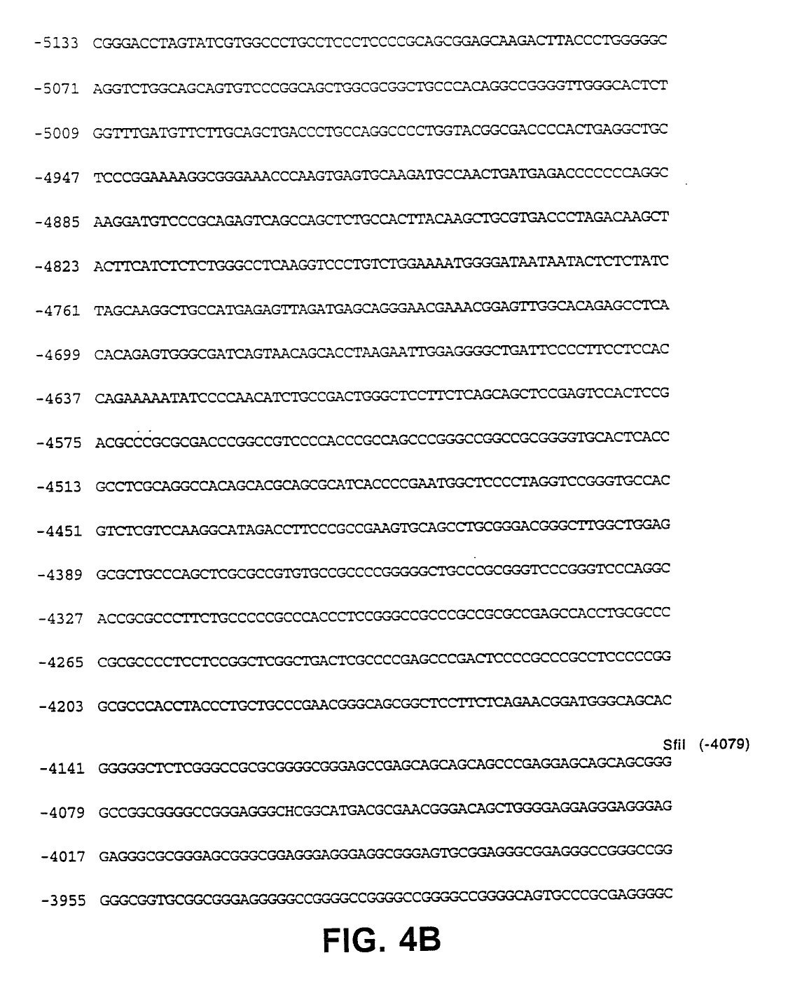

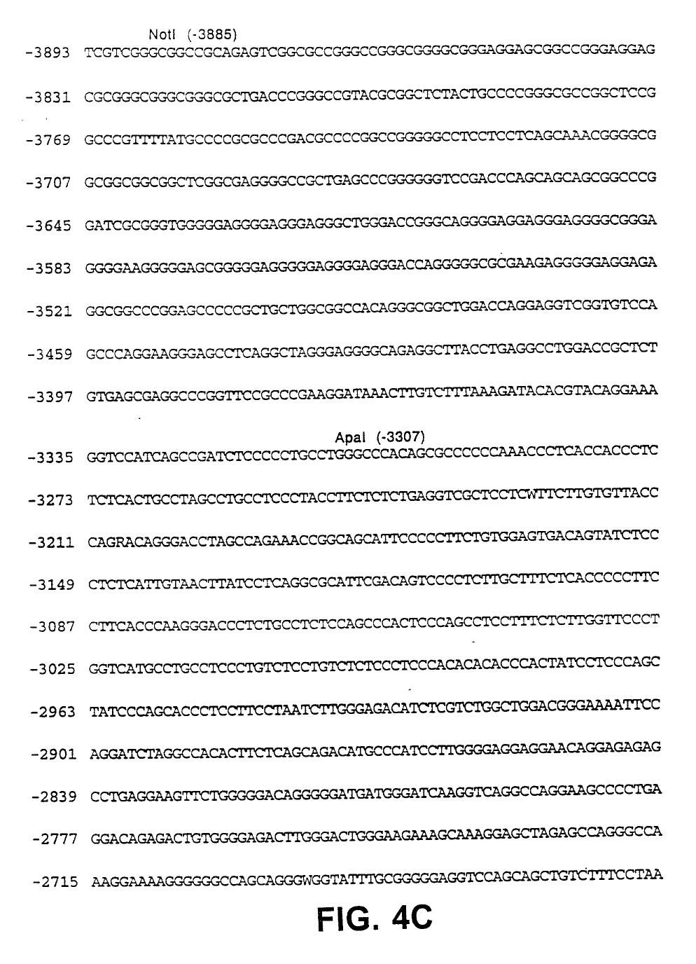

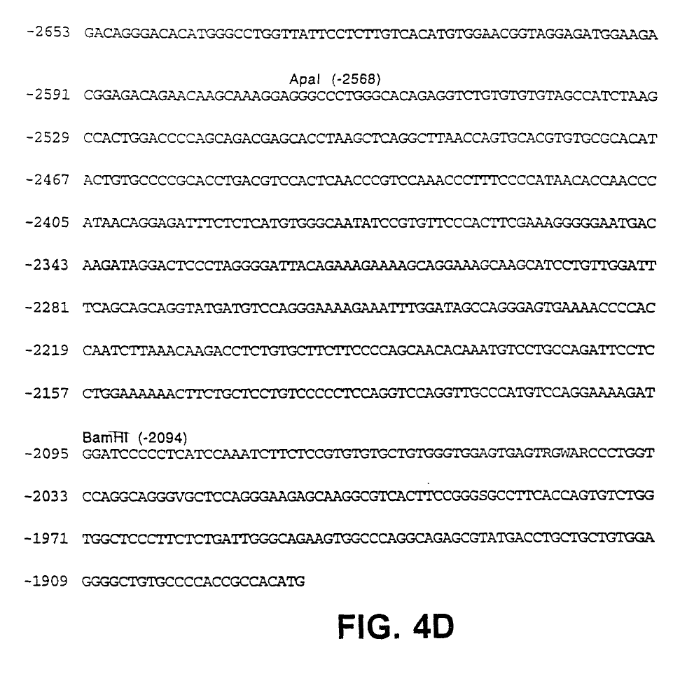

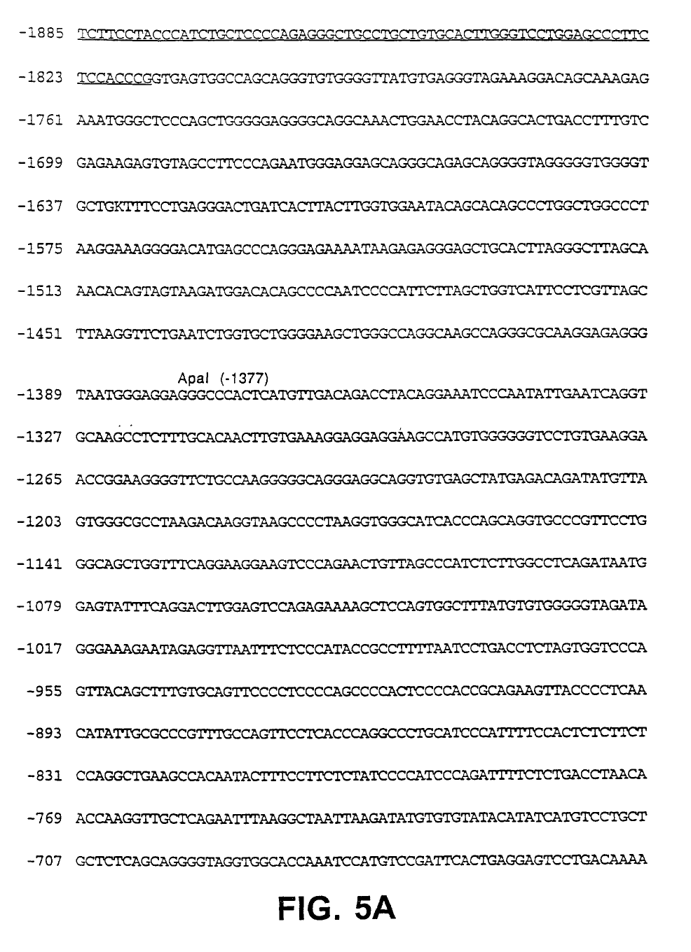

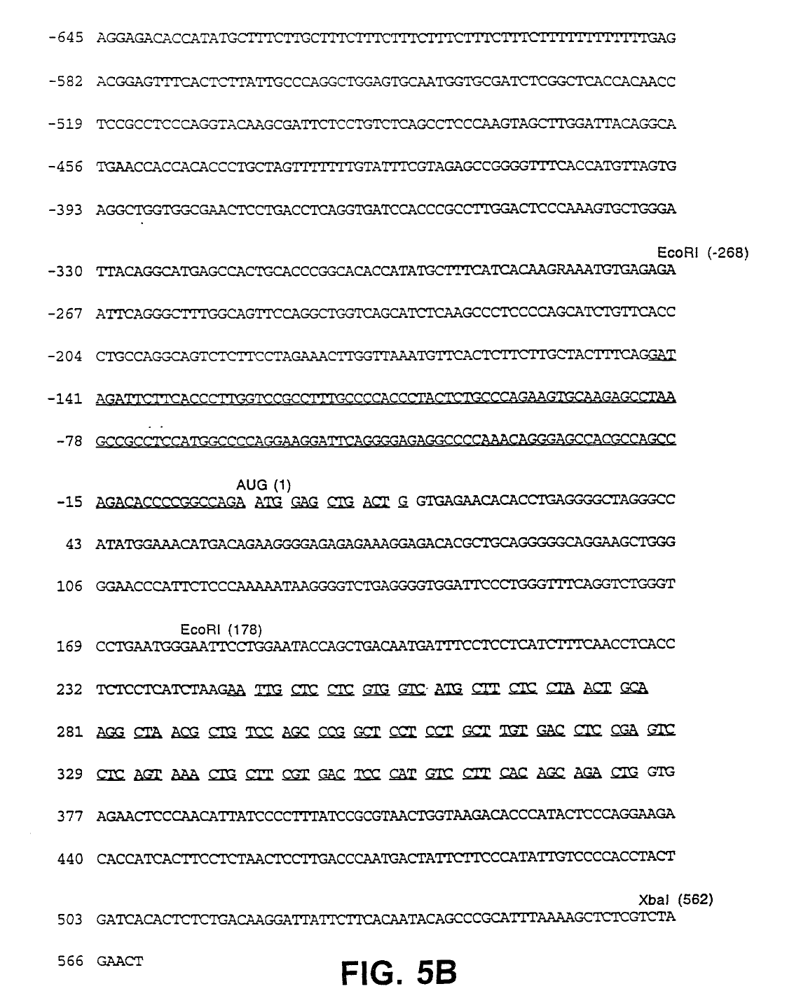

Analysis of the genomic DNA sequences and comparison to the known cDNA sequences revealed features essential for the construction of targeting constructs. For example, for the first time, it is shown that the first exon of the human TPO gene is entirely non-coding, and that translation initiates within the second exon of the endogenous gene. This information was important to the design of the gene activation constructs described herein, in which splicing of an exogenous exon to the endogenous second exon requires that the exogenous exon be non-coding, or in which splicing of an exogenous coding exon requires that targeting be performed such that the exogenous coding exon is inserted in a position so that it can be spliced to the endogenous third exon of the TPO gene. Furthermore, the cloning of approximately 6.3 kb of DNA sequence from upstream of the human TPO gene provided targeting sequences useful for the development of gene activation constructs. FIG. 4 shows approximately 4.5 kb of novel DNA sequence from the human TPO locus lying 5′ of the known cDNA sequence (de Sauvage, F. J. et al., op. cit.). FIG. 5 shows approximately 2.5 kb of DNA sequence from the human TPO locus extending in the 3′ direction from the 5′ boundary of the known cDNA sequence. Intron sequences (positions −1815 to −145, positions 14 to 245, and positions 374 to 570) of FIG. 5 are novel. DNA constructs comprising the novel sequences of FIGS. 4 and 5, or fragments derived from these sequences, are useful for homologous recombination as taught herein.

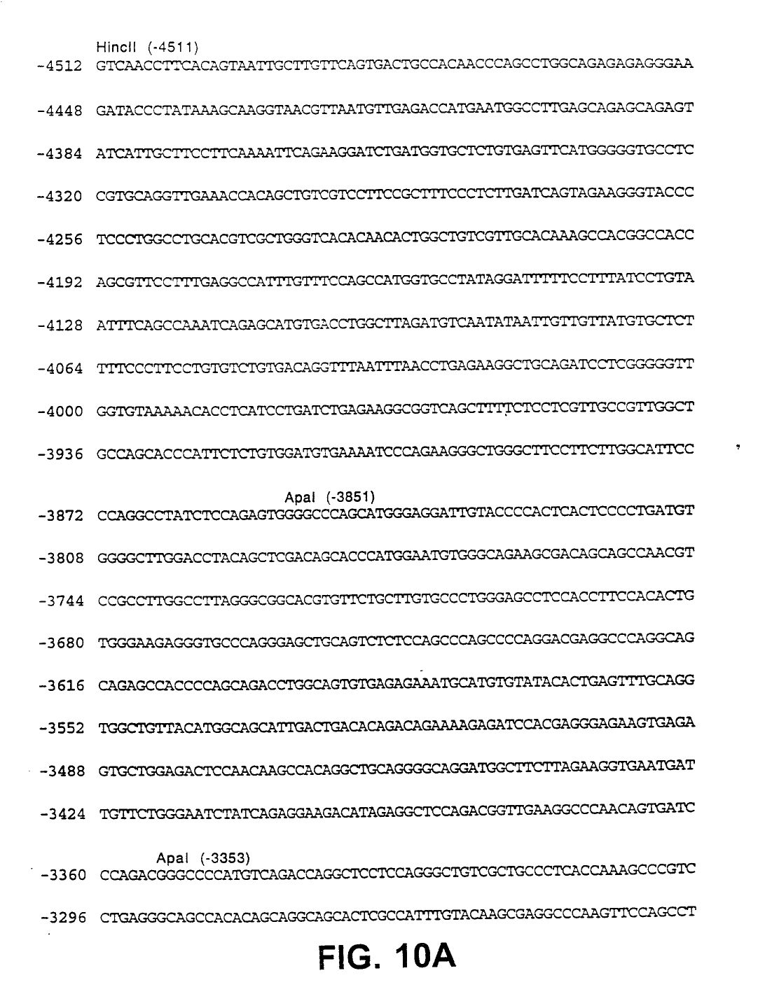

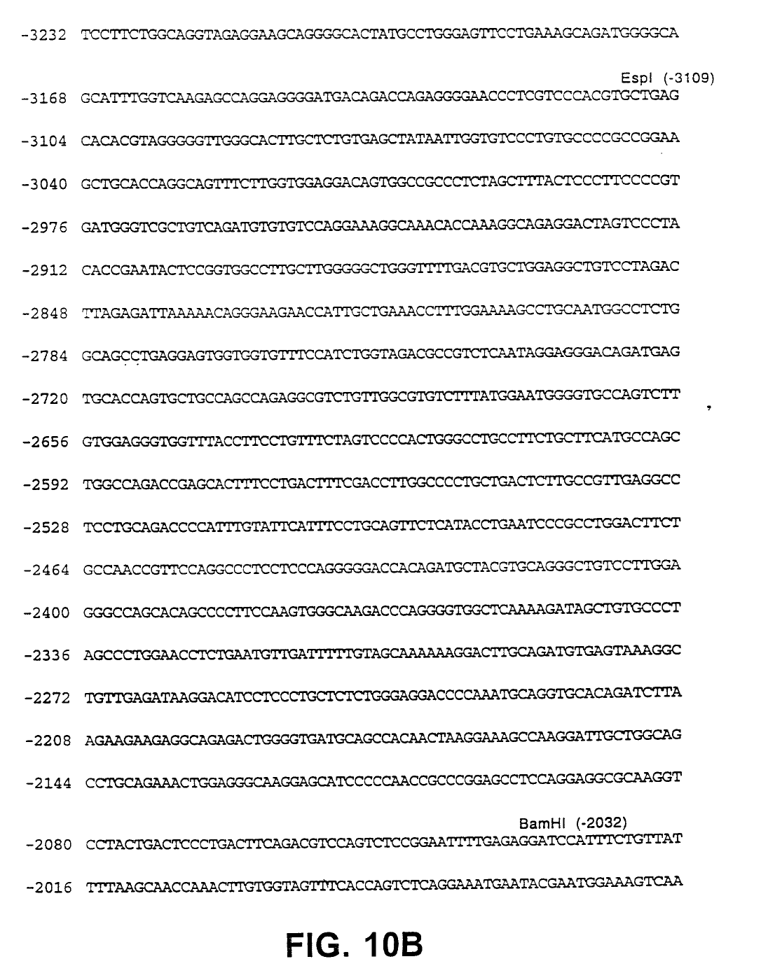

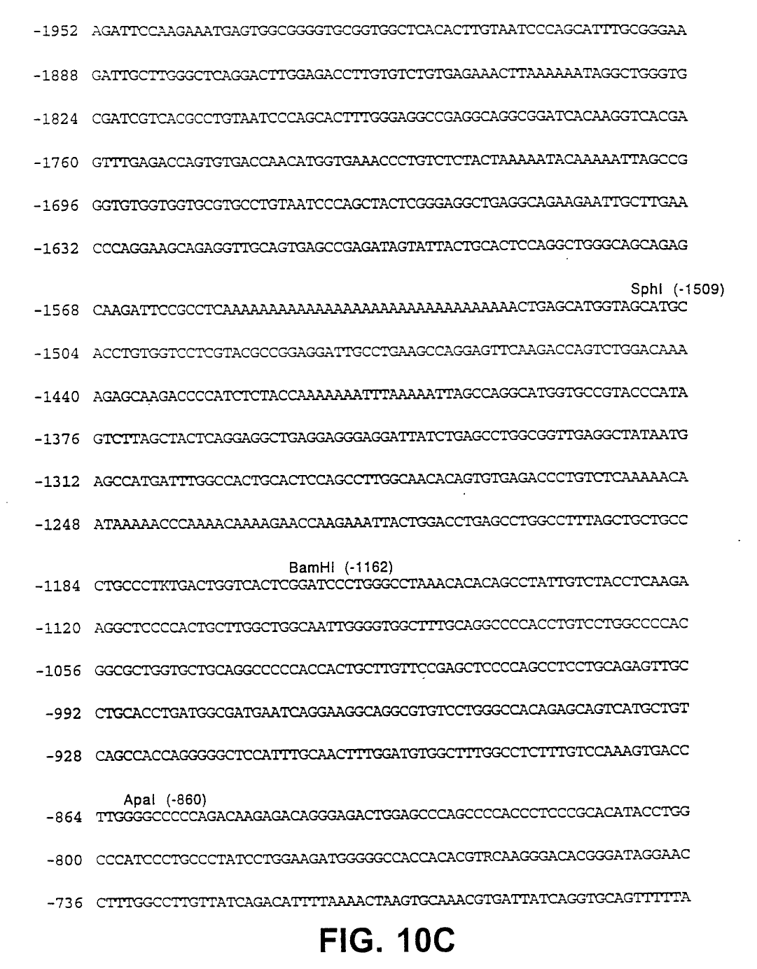

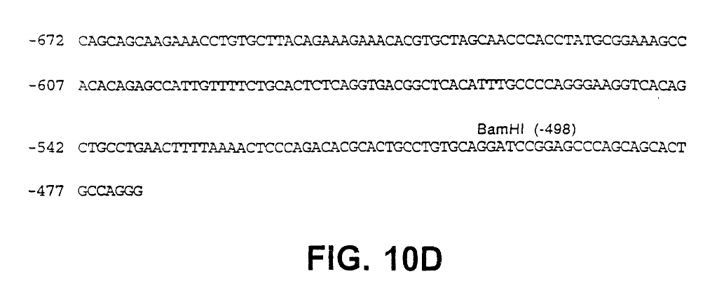

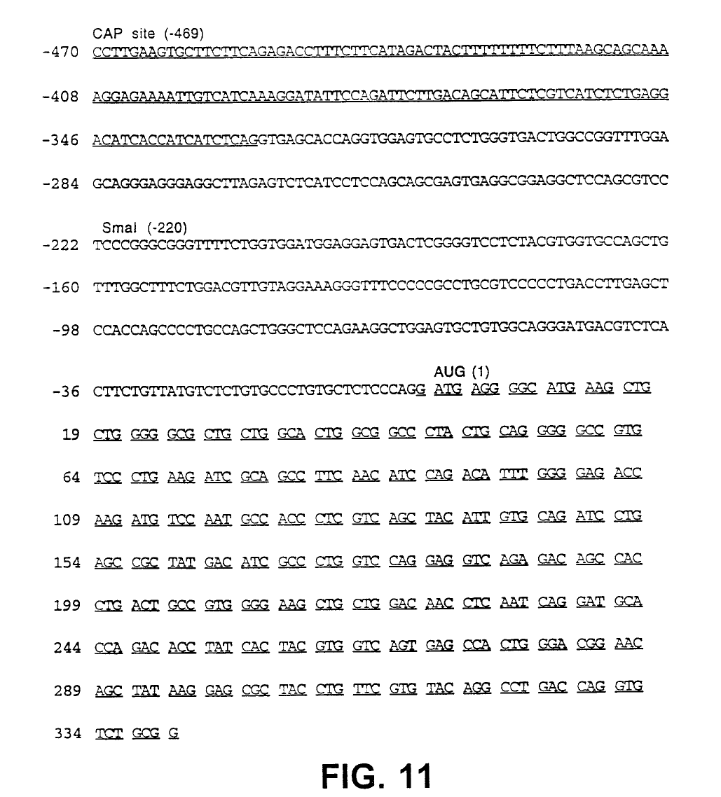

Similarly, for the first time it is shown that the first exon of the human DNase I gene is entirely non-coding. This information was important to the design of the targeting constructs described herein. Example 5, for example, describes a targeting construct which includes two non-coding exons separated by an intron, and which is inserted upstream of DNase I exon 1. This configuration allows promoter position to be optimized by varying the length of either the exogenous intron or the intron present between the exogenous exon and the endogenous second exon of the DNase I gene, while ensuring that the primary transcript will be spliced appropriately and that translation initiates at the correct position for synthesis of functional DNase I. Furthermore, the cloning of approximately 4.5 kb of DNA sequence from upstream of the human DNase I gene provided targeting sequences useful for the development of gene activation constructs. FIG. 10 shows approximately 4 kb of novel DNA sequence from the human DNase I locus lying 5′ of the known cDNA sequence (Shak, S. et al. op. cit.). FIG. 11 shows approximately 0.8 kb of DNA sequence from the human DNase I locus extending in the 3′ direction from the 5′ boundary of the known cDNA sequence. Intron sequences (positions −328 to −2) of FIG. 11 are novel. DNA constructs comprising the novel sequences of FIGS. 10 and 11, or fragments derived from these sequences, are useful for homologous recombination as described herein.

Finally, the analysis of the upstream region of the β-interferon gene (a gene which is known to lack introns) was cloned and sequenced and a detailed restriction map was produced. Previously, only 357 bp of DNA upstream of the translation initiation codon was characterized (see Genbank entry HUMIFNB1F). The cloning and sequence analysis provided approximately 9.6 kb of genomic DNA upstream of the gene for the design and construction of a targeting construct (Example 7). FIG. 14 shows approximately 8.4 kb of novel DNA sequence from the β-interferon locus lying 5′ of the known sequences (Genbank entry HUMIFNB1F). DNA constructs comprising the novel sequences of FIG. 14, or fragments derived from these sequences, are useful for homologous recombination as taught herein.

The following defines the DNA constructs of the present invention, the elements comprising the DNA constructs of the present invention (Section A), methods in which the DNA constructs are used to produce homologously recombinant cells (Section B), the structure of the targeted gene and the resulting product (Section C), the homologously recombinant cells produced (Section D), uses of these cells (Sections E and F), and the advantages of the constructs and methods described herein (Section G).

A. The DNA Construct

The DNA constructs of the present invention include at least the following components: a targeting sequence; a regulatory sequence; an exon and a splice-donor site. In the construct, the exon is 3′ of the regulatory sequence and the splice-donor site is 3′ of the exon. In addition, there can be multiple exons and/or introns preceding (5′ to) the exon flanked by the splice-donor site. Taken as a group, the exons, introns, and splice-sites are referred to as the “structural elements” of the construct, so-called because they are important in defining the structure of the novel gene produced by homologous recombination between genomic DNA and DNA of the targeting construct. As described herein, there frequently are additional construct components, such as a selectable and/or amplifiable markers.

The DNA in the construct is referred to as exogenous DNA, defined herein as DNA which is introduced into a cell by the methods described herein, such as with the DNA constructs of the present invention. Exogenous DNA can contain sequences identical to or different from the endogenous DNA. The term endogenous DNA is defined herein as DNA present in the cell as obtained.

The DNA of the construct can be obtained from sources in which it occurs in nature or can be produced, using genetic engineering techniques or synthetic processes.

1. The Targeting Sequence

The targeting sequence or sequences are DNA sequences which permit homologous recombination into the genome of the selected cell containing the gene of interest. Targeting sequences are, generally, DNA sequences which are homologous to (i.e., identical or sufficiently similar to) DNA sequences present in the genome of the cells as obtained (e.g., coding or noncoding DNA, located upstream of the transcriptional start site, within the transcribed region encompassing the gene, or downstream of the transcriptional stop site of the gene, or sequences present in the genome through a previous modification), such that the targeting sequence and cellular DNA can undergo homologous recombination. In general, two sequences are described as homologous if a DNA strand of one sequence is capable of hybridizing to a DNA strand of the other sequence under conditions standardly used for the detection of sequence similarity (see, for example, Ausubel et al., Current Protocols in Molecular Biology, Wiley, New York, N.Y. (1987)). The targeting sequence or sequences used are selected with reference to the site into which the DNA in the DNA construct is to be inserted and may be derived from either genomic or cDNA sequences. Typically, a targeting sequence is at least about 20 base pairs in length. The size of the sequence is chosen to be a size which selectively promotes homologous recombination with desired genomic DNA sequences.

One or more targeting sequences can be employed. For example, a circular plasmid or DNA fragment preferably employs a single targeting sequence. A linear plasmid or DNA fragment preferably employs two targeting sequences with exogenous DNA to be inserted into genome positioned between the two targeting sequences. The targeting sequence or sequences can be within an endogenous gene (e.g., within the sequences of an exon and/or intron), within the endogenous promoter sequences, or upstream of the endogenous promoter sequences. The targeting sequence or sequences can include those regions of a gene presently known or sequenced and/or regions further upstream which are structurally uncharacterized but can be mapped using restriction enzymes and cloning approaches available to one skilled in the art.

2. The Regulatory Sequence

The regulatory sequence of the DNA construct can be comprised of one or more of a variety of elements, including: promoters (such as a constitutive or inducible promoters), enhancers, scaffold-attachment regions or matrix attachment regions, (McKnight, R. A. et al., Proc. Natl. Acad. Sci. USA 89:6943-6947 (1992); Phi-Van, L. and Stratling, W. H. EMBO J. 7:655-664 (1988)) negative regulatory elements, locus control region, (Pondel, M. D. et al., Nucl. Acids Res. 20:237-243 (1992); Li, Q. and Stamatoyannopoulos, G. Blood 84:1399-1401 (1994)) transcription factor binding sites, or combinations of said sequences.

3. Structural Elements of the DNA Construct

a. Exons and Introns

An exon is defined herein as a DNA sequence which is copied into RNA and is present in a mature mRNA molecule. An intron is defined as a sequence of one or more nucleotides lying between two exons and which is removed, by splicing, from a precursor RNA molecule in the formation of an mRNA molecule.

The DNA constructs of the present invention contain one or more exons. The exons can, optionally, contain DNA which encodes one or more amino acids and/or partially encodes an amino acid (i.e., one or two bases of a codon). Where the exogenous exon or exons encode one or more amino acids and/or a portion of an amino acid, the DNA construct is designed such that, upon transcription and splicing, the reading frame is in-frame with the second or subsequent exon of the endogenous gene's coding region. As used herein, in-frame means that the encoding sequences of, for example, a first exon and a second exon when fused, join together nucleotides in a manner that does not change the appropriate reading frame of the portion of the mRNA derived from the second exon.

In the case of activating the TPO and DNase I genes, the exogenous exon can, preferably, be derived from any gene in which the exon includes a CAP site and non-coding sequences. Examples would include the first exon of the CMV immediate-early gene and follicle stimulating hormone (FSH) gene. In the case of β-interferon, whose gene contains no natural introns, there are preferably two exogenous non-coding exons, separated by an intron, in the targeting construct.

b. Splice-Sites

Introns contained within the mRNA of eukaryotic cells are removed through the recognition of signals termed splice-donor and splice-acceptor sites. A splice-donor site is a sequence which directs the splicing of one exon to another exon. Typically, the first exon lies 5′ of the second exon, and the splice-donor site overlapping and flanking the first exon on its 3′ side recognizes a splice-acceptor site flanking the second exon on the 5′ side of the second exon. Splice-donor sites have a characteristic consensus sequence represented as: (A/C)AGGURAGU (where R denotes a purine nucleotide) with the GU in the fourth and fifth positions being required (Jackson, I. J., Nucleic Acids Research 19: 3715-3798 (1991)). The first three bases of the splice-donor consensus site are the last three bases of the exon. Splice-donor sites are functionally defined by their ability to effect the appropriate reaction within the mRNA splicing pathway.

An unpaired splice-donor site is defined herein as a splice-donor site which is present in a targeting construct and is not accompanied in the targeting construct by a splice-acceptor site positioned 3′ to the unpaired splice-donor site. Upon homologous recombination between the targeting sequences and genomic DNA, the unpaired splice-donor site results in splicing to an endogenous splice-acceptor site.

A splice-acceptor site is a sequence which, like a splice-donor site, directs the splicing of one exon to another exon. Acting in conjunction with a splice-donor site, the splicing apparatus uses a splice-acceptor site to effect the removal of an intron. Splice-acceptor sites have a characteristic sequence represented as: YYYYYYYYYYNYAG, where Y denotes any pyrimidine and N denotes any nucleotide (Jackson, I. J., Nucleic Acids Research 19:3715-3798 (1991)).

c. Marker Genes for Selection and Amplification

The identification of the targeting event can be facilitated by the use of one or more selectable marker genes typically contained within the targeting DNA construct. The use of both positively and negatively selectable markers for identifying targeted events is described in related pending applications U.S. Ser. No. 08/243,391, U.S. Ser. No. 07/985,586, U.S. Ser. No. 07/789,188, PCT/US93/11704, and PCT/US92/09627.

Homologously recombinant cells containing multiple copies of the novel transcription units produced by the present invention may be isolated by including within the targeting DNA construct an amplifiable marker gene which has the property that cells containing multiple copies of the selectable marker gene can be selected for by culturing the cells in the presence of an appropriate selectable agent. The novel transcription unit will be amplified in tandem with the amplified selectable marker gene, allowing the production of very high levels of the desired protein. Amplifiable marker genes and their use are described in applications U.S. Ser. No. 08/243,391, U.S. Ser. No. 07/985,586, and PCT/US93/11704.

In one embodiment the positively selectable marker neo is used (derived from the bacterial neomycin phosphotransferase gene) is used to select for cells which have stably incorporated the DNA of the targeting construct, and the mouse dhfr (dihydrofolate reductase) gene is used to subsequently amplify the novel transcription unit present in homologously recombinant cells.

d. Additional Elements of the Targeting Construct

As taught herein, gene targeting can be used to insert a regulatory sequence within an endogenous gene (e.g., within the sequences of an exon and/or intron), within the endogenous promoter sequences, or upstream of the endogenous promoter sequences, with said genes corresponding to the endogenous cellular TPO, β-interferon, or DNase I gene. Alternatively or additionally, the targeting constructs may be designed to include sequences which affect the structure or stability of the TPO, β-interferon, or DNase I protein or corresponding RNA molecule. For example, RNA stability elements, splice sites, and/or leader sequences of RNA molecules can be modified to improve or alter the function, stability, and/or translatability of an RNA molecule. Protein sequences may also be altered, such as signal sequences, active sites, and/or structural sequences for enhancing or modifying glycosylation, transport, secretion, or functional properties of a protein. According to this method, introduction of the exogenous DNA results in the alteration of the structural or functional properties of the expressed proteins or RNA molecules.

In one embodiment the method can be used to create novel transcription units encoding fusion proteins in which structural, enzymatic, or ligand or receptor binding protein domains of another protein are fused to TPO, DNase I, or β-interferon. In these cases the exogenous coding DNA contains an ATG translation initiation codon in-frame with the coding sequences of the endogenous TPO, DNase I, or β-interferon gene. For example, the exogenous DNA can encode a sequence which can anchor TPO or DNase I to a membrane, a portion of a signal peptide designed to improve cellular secretion, leader sequences, enzymatic regions, transmembrane domain regions, co-factor binding regions, or other functional regions.

The DNA construct can also include a bacterial origin of replication and bacterial antibiotic resistance markers or other selectable markers, which allow for large-scale plasmid propagation in bacteria or any other suitable cloning/host system.

B. Transfection and Homologous Recombination

According to the present method, the construct is introduced into the cell, such as a primary, secondary, or immortalized cell, as a single DNA construct, or as separate DNA sequences which become incorporated into the chromosomal or nuclear DNA of a transfected cell.

The targeting DNA construct can be introduced into cells on a single DNA construct or on separate constructs. The total length of the DNA construct will vary according to the number of components and the length of each and the construct will generally be at least about 200 nucleotides. Further, the DNA can be introduced as linear, double-stranded (with or without single-stranded regions at one or both ends), single-stranded, or circular DNA.

Any of the construct types of the disclosed invention is then introduced into the cell to obtain a transfected cell. The transfected cell is maintained under conditions which permit homologous recombination, as is known in the art (reviewed in Capecchi, M. R., Science 244:1288-1292 (1989)). When the homologously recombinant cell is maintained under conditions sufficient for transcription of the DNA, the regulatory region introduced by the targeting construct, as in the case of a promoter, will activate expression of the novel transcription unit produced by homologous recombination.

The DNA constructs may be introduced into cells by a variety of physical or chemical methods, including electroporation, microinjection, microprojectile bombardment, calcium phosphate precipitation, and liposome-, polybrene-, or DEAE dextran-mediated transfection.

C. The Targeted Gene and Resulting Product

The targeting DNA construct, when introduced by homologous recombination or targeting into cells containing the TPO, β-interferon, or DNase I gene, produces a novel transcription unit which results in the expression of TPO, β-interferon, or DNase I.

At the targeted site in the genome, the exogenous regulatory sequence is operatively linked to a CAP site, which initiates transcription. Operatively linked is defined as a configuration in which the exogenous regulatory sequence, exon, splice-donor site and, optionally, an intron sequence and splice-acceptor site, are appropriately targeted at a position relative to the endogenous gene such that the regulatory element directs the production of a primary RNA transcript which initiates at a CAP site and includes sequences corresponding to the exogenous exon or exons and endogenous exons the TPO, DNase I, or β-interferon gene. In an operatively linked configuration the splice-donor site of the targeting construct directs a splicing event between an exogenous exon and the splice-acceptor site of an endogenous exon, such that a desired protein can be produced from the fully spliced mature transcript. In one embodiment, the splice-acceptor site is endogenous, such that the splicing event is directed to an endogenous exon of the TPO or DNase I gene. In another embodiment an intron and a splice-acceptor site are included in the targeting construct used to activate the β-interferon gene, and a splicing event removes the intron introduced by the targeting construct.

D. The Homologously Recombinant Cells

The targeting event results in the insertion of the regulatory and structural sequences of the targeting construct into a cell's genome, creating a novel transcriptional unit under the control of the exogenous regulatory sequences.

Homologous recombination between the genomic DNA and the introduced DNA results in a homologously recombinant cell, which may be a primary, secondary, or immortalized human or other mammalian cell in which sequences which alter the expression of an endogenous gene are operatively linked to the endogenous TPO, DNase I, or β-interferon gene. Particularly, the invention includes a homologously recombinant cell comprising exogenous regulatory sequences and an exon, flanked by a splice-donor site, which are introduced at a predetermined site by a targeting DNA construct, and are operatively linked to the coding region of the endogenous gene. Optionally, there may be multiple exogenous exons (coding or non-coding) and introns operatively linked to any exon of the endogenous gene. The resulting homologously recombinant cells are cultured under conditions which select for amplification, if appropriate, of the DNA encoding the amplifiable marker and the novel transcriptional unit. With or without amplification, cells produced by this method can be cultured under conditions, as are known in the art, suitable for the expression of TPO, β-interferon, or DNase I.

The targeting constructs and methods of the present invention may be used with, for example, primary or secondary cell strains (which exhibit a finite number of mean population doublings in culture and are not immortalized) and immortalized cell lines (which exhibit an apparently unlimited lifespan in culture). Primary and secondary cells include, for example, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. Where the homologously recombinant cells are to be used in gene therapy, primary cells are preferably obtained from the individual to whom the resulting homologously recombinant cells are administered. However, primary cells can be obtained from a donor (other than the recipient) of the same species. Examples of immortalized human cell lines which may be used with the DNA constructs and methods of the present invention include, but are not limited to, HT1080 cells (ATCC CCL 121), HeLa cells and derivatives of HeLa cells (ATCC CCL 2, 2.1 and 2.2), MCF-7 breast cancer cells (ATCC BTH 22), K-562 leukemia cells (ATCC CCL 243), KB carcinoma cells (ATCC CCL 17), 2780AD ovarian carcinoma cells (Van der Blick, A. M. et al., Cancer Res, 48:5927-5932 (1988), Raji cells (ATCC CCL 86), WiDr colon adenocarcinoma cells (ATCC CCL 218), SW620 colon adenocarcinoma cells (ATCC CCL 227), Jurkat cells (ATCC TIB 152), Namalwa cells (ATCC CRL 1432), HL-60 cells (ATCC CCL 240), Daudi cells (ATCC CCL 213), RPMI 8226 cells (ATCC CCL 155), U-937 cells (ATCC CRL 1593), Bowes Melanoma cells (ATCC CRL 9607), WI-38VA13 subline 2R4 cells (ATCC CLL 75.1), and MOLT-4 cells (ATCC CRL 1582), as well as heterohybridoma cells produced by fusion of human cells and cells of another species. Secondary human fibroblast strains, such as WI-38 (ATCC CCL 75) and MRC-5 (ATCC CCL 171) may be used. Further discussion of the types of cells that may be used in practicing the methods of the present invention is presented in applications U.S. Ser. No. 08/243,391, U.S. Ser. No. 07/985,586, U.S. Ser. No. 07/789,188, U.S. Ser. No. 07/911,533, U.S. Ser. No. 07/787,840, PCT/US93/11704, and PCT/US92/09627.

E. In vivo Protein Production

Homologously recombinant cells of the present invention in which the expression properties of the endogenous TPO, β-interferon, or DNase I gene are altered are useful in gene therapy, as populations of homologously recombinant cell lines, as populations of homologously recombinant primary or secondary cells, homologously recombinant clonal cell strains or lines, homologously recombinant heterogenous cell strains or lines, and as cell mixtures in which at least one representative cell of one of the preceding categories of homologously recombinant cells is present. Homologously recombinant primary cells, clonal cell strains or heterogenous cell strains are administered to an individual in whom the abnormal or undesirable condition is to be treated or prevented, in sufficient quantity and by an appropriate route, to express or make available the desired product at physiologically relevant levels. A physiologically relevant level is one which either approximates the level at which the product is normally produced in the body or results in improvement of the abnormal or undesirable condition. Methods for gene therapy in which homologously recombinant cells are introduced into an individual for the purpose of in vivo protein production are described in pending applications U.S. Ser. No. 08/243,391, U.S. Ser. No. 07/985,586, U.S. Ser. No. 07/789,188, U.S. Ser. No. 07/911,533, U.S. Ser. No. ______, PCT/US93/11704, and PCT/US92/09627.

In one embodiment, the invention relates to a method of providing TPO to a mammal introducing homologously recombinant cells into the mammal in sufficient number to produce an effective amount of TPO in the mammal.

In another embodiment homologously recombinant cells expressing DNase I can be administered to the trachea and lungs of a cystic fibrosis patient, for the purpose of in vivo secretion of DNase I for the relief of respiratory distress.

In a third embodiment, homologously recombinant cells expressing β-interferon may be implanted into a patient suffering from multiple sclerosis, for the purpose of in vivo secretion of β-interferon to diminish exacerbations associated with the disease.

F. In vitro Protein Production

Homologously recombinant cells produced according to this invention can also be used for in vitro production of TPO, β-interferon, or DNase I. The cells are maintained under conditions, as are known in the art, which result in expression of the protein. Proteins expressed using the methods described may be purified from cell lysates or cell supernatants. Proteins made according to this method can be prepared as a pharmaceutically-useful formulation and delivered to a human or non-human animal by conventional pharmaceutical routes as is known in the art (e.g., oral, intravenous, intramuscular, intranasal, intratracheal or subcutaneous). As described herein, the homologously recombinant cells can be immortalized, primary, or secondary human cells. The use of cells from other species may be desirable in cases where the non-human cells are advantageous for protein production purposes where the non-human TPO, DNase I, or β-interferon produced is useful therapeutically.

G. Advantages

The methodologies, DNA constructs, cells, and resulting proteins of the invention herein possess versatility and many other advantages over processes currently employed within the art in gene targeting. The ability to activate expression of an endogenous TPO, β-interferon, or DNase I gene by positioning an exogenous regulatory sequence and other structural sequences at various positions ranging from directly fused to portions of the normal gene's coding region to 30 kilobase pairs or further upstream of the transcribed region of an endogenous gene, or within an intron of an endogenous gene, is advantageous for gene expression in cells. For example, it can be employed to position the regulatory element upstream or downstream of regions that normally silence or negatively regulate a gene. The positioning of a regulatory element upstream or downstream of such a region can override such dominant negative effects that normally inhibit transcription. In addition, regions of DNA that normally inhibit transcription or have an otherwise detrimental effect on the expression of a gene may be deleted using the targeting constructs, described herein. The present invention also allows proteins to be expressed in the context of their normal intron sequences, which have been shown to be important factors in the expression of genes in mammalian cells (cf. Korb. M. et al. Nucl. Acids Res. 21: 5901-5908 (1993)).

Additionally, since promoter function is known to depend strongly on the local environment, a wide range of positions may be explored in order to find those local environments optimal for function. However, since, ATG start codons are found frequently within mammalian DNA (approximately one occurrence per 48 base pairs as calculated from nearest-neighbor dinucleotide frequencies in human DNA), transcription cannot simply initiate at any position upstream of a gene and produce a transcript containing a long leader sequence preceding the correct ATG start codon, since the frequent occurrence of ATG codons in such a leader sequence will prevent translation of the correct gene product and render the message useless. Thus, the incorporation of an exogenous exon, a splice-donor site, and, optionally, an intron and a splice-acceptor site into targeting constructs comprising a regulatory region allows gene expression to be optimized by identifying the optimal site for regulatory region function, without the limitation imposed by needing to avoid inappropriate ATG start codons in the mRNA produced. This provides significantly increased flexibility in the placement of the construct and makes it possible to activate a wider range of genes than is possible using other technologies. For example, U.S. Pat. No. 5,272,071 and foreign patent applications WO 91/06666, WO 91/06667 and WO 90/11354 describe homologous recombination methods for inserting a regulatory sequence upstream of the coding region of an endogenous gene. In these methods, only a very small number of positions for promoter insertion are acceptable for expression, limited by the frequent occurrence of ATG start codons as described above.

The present invention provides further advantages over the methods available in the art. For example, the use of homologous recombination results in the production of cells in which the novel transcription unit is present in the same location in all cells in which homologous recombination has occurred. Thus, the novel transcription unit will function similarly in all homologously recombinant cells derived independently. This allows for the production of cells with highly predictable properties. In the case of in vitro protein production, it is desirable to develop cells in which the behavior (e.g. the expression and amplification properties) of the desired gene can be controlled and there is little variation when comparing individual cells which are being processed for large-scale production purposes. In the case of in vivo protein production or gene therapy, it is desirable to be able to develop cells in which the properties are predictable and uniform among individual patients. This allows for a high degree of precision in achieving appropriate levels of the desired protein in vivo, leading to controlled and reproducible methods for treating disease.

The DNA constructs described above are useful for operatively linking exogenous regulatory and structural elements to endogenous coding sequences in a way that precisely creates a novel transcriptional unit, provides flexibility in the relative positioning of exogenous regulatory elements and endogenous genes and, ultimately, enables a highly controlled system for and regulating expression of genes of therapeutic interest.

The subject invention will now be illustrated by the following examples, which are not intended to be limiting in any way.

### EXAMPLES

### Example 1

### Cloning of the TPO Gene and Identification of 5′ Flanking Sequences

The human thrombopoietin gene was isolated from a human genomic DNA library. The library was prepared from male leukocyte DNA partially-digested with MboI and cloned into the bacteriophage vector lambda EMBL3 (Clontech, Palo Alto, Calif.; Cat. #HL1006d). For screening, a probe was isolated by PCR amplification of human genomic DNA using oligonucleotides 1.1 and 1.2.

| Oligo 1.1 (TPO sense) | (SEQ ID NO: 1) |
|---|---|
| 5′ AATTGCTCCT CGTGGTCATG CTTCT | |
| Oligo 1.2 (TPO anti-sense) | (SEQ ID NO: 2) |

5′ CTGTGAAGGA CATGGGAGTC A

These primers were designed using the known TPO mRNA sequence (de Sauvage, F. J. et al. Nature 369:533-538 (1994)). The amplified probe (probe A; 120 bp) was labeled with ³²P dCTP by the polymerase chain reaction and used to screen the genomic DNA library. Filters were hybridized for 6 hours at 68° C. in 125 mM Na₂HPO₄ (pH 7.2), 250 mM NaCl, 10% PEG 8000, 7% SDS, 1 mM EDTA. Filters were washed twice in 500 ml of 20 mM Na₂HPO₄₁ (pH 7.2), 1 mM EDTA, 5% SDS, followed by 4 washes in 500 ml of 20 mM Na₂HPO₄, (pH 7.2), 1 mM EDTA, 1% SDS. The wash buffers were pre-heated to 56° C. and washing was done on a rotary shaker at room temperature for approximately 5 minutes per wash. The hybridizing signals were identified by autoradiography at −80° C. with an intensifying screen. In one experiment, approximately 1.4×10⁶ phage were screened and 7 positive signals were obtained. Phage plaques corresponding to positive signals were plaque purified. Following 2 rounds of plaque purification by low density screening using probe A, 4 of the phage, designated 5B, 25A, 25B and 28B, were retained for further analysis. Plaque purified phage were amplified and isolated by cesium chloride gradient ultracentrifugation (Yamamoto K. R. et al., Virology 40:734 (1970)) and DNA was isolated. Library screening, plaque purification of recombinant bacteriophage, and isolation bacteriophage DNA was performed using standard methods (Ausubel et al., Current Protocols in Molecular Biology, Wiley, New York, N.Y. (1987)).

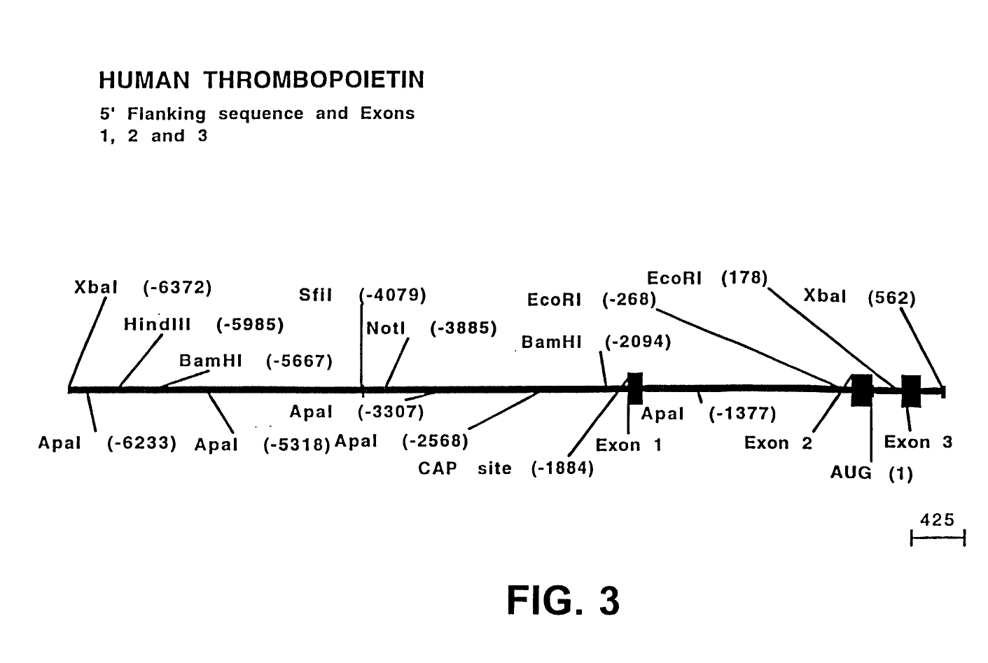

An approximately 6.9 kb XbaI fragment comprising exon 1, intron 1, exon 2, intron 2, exon 3, and a portion of intron 3, as well as approximately 4.3 kb of nontranscribed DNA lying upstream of TPO exon 1 was identified by restriction enzyme and Southern hybridization analysis using probe A. This fragment was isolated from one genomic clone (28B) and subcloned into plasmid pBSIISK⁺ (Stratagene Inc., La Jolla, Calif.) for further analysis. The resultant clones, pBS(X)/5′Thromb.8 and pBS(X)/5′Thromb.2, harbor the 6.9 kb XbaI fragment in opposite orientations with respect to the plasmid backbone. Restriction enzyme mapping yielded the restriction enzyme map shown in FIG. 3. The nucleotide sequence of the portion of this fragment lying upstream of the 5′ end of the known cDNA sequence is shown in FIG. 4 (SEQ ID NO: 3). The nucleotide sequence of the portion of the 6.9 kb XbaI fragment lying downstream of the 5′ end of the known cDNA sequence is shown in FIG. 5 (SEQ ID NO: 4). Comparison of the cloned genomic sequence presented here with the published cDNA sequence (de Sauvage, F. J. et al. Nature 369:533-538 (1994)) reveals that the 5′ end of the TPO gene consists of a non-coding exon (exon 1) of at least 107 bp, a second exon (exon 2) which is 158 bp, and a third exon (exon 3) which is 128 bp in length. The 13 base pairs at the 3′ end of exon 2 code for the first four and a portion of the fifth amino acid of the TPO signal peptide. Exon 3 codes for the remainder of the 21 amino acid signal peptide and a portion of the mature TPO polypeptide. Exons 1 and 2 are separated by intron 1 (1671 bp), and exons 2 and 3 are separated by intron 2 (231 bp). There are two differences between the sequence reported in FIG. 5 and the sequence published by de Sauvage et al.: nucleotides at positions −134 and −124 are reported as C residues by de Sauvage et al. and are shown as T residues in FIG. 5. These residues are outside of the coding sequence for TPO and may be explained by sequence polymorphism or by errors in compilation of the published sequence. In any event, this minor difference does not impact the ability of the person of skill to practice the invention as described herein.

### EXAMPLE 2

### Construction of Targeting Plasmids for Activation and Amplification of the TPO Gene

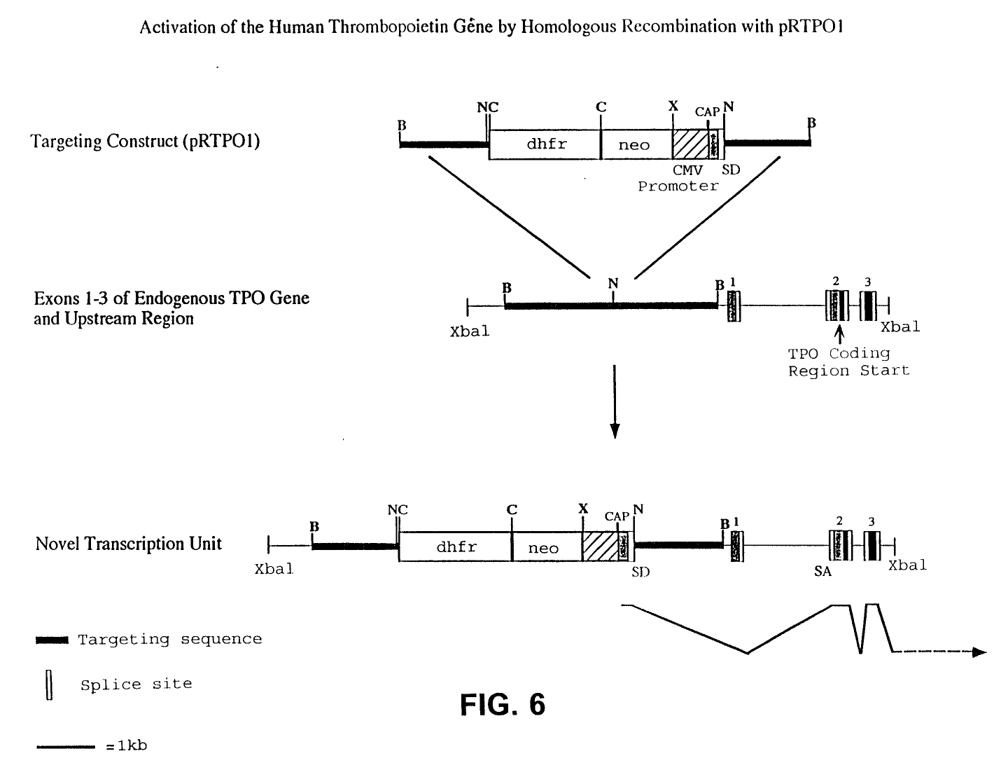

The activation of the TPO gene can be accomplished by a number of strategies, as shown in FIGS. 6-8. In the strategy shown in FIG. 6, a targeting fragment is introduced into the genome of recipient cells for insertion of a regulatory region, a non-coding exon, and a functional, unpaired splice-donor site upstream of the TPO coding region. Specifically, the targeting construct from which this fragment is derived (pRTPO1) is designed to include a first targeting sequence homologous to sequences upstream of the TPO gene, an amplifiable marker gene, a selectable marker gene, a regulatory region, a CAP size, a non-coding exon, an unpaired splice-donor site, and a second targeting sequence corresponding to sequences downstream of the first targeting sequence but upstream of TPO exon 1. By this strategy, homologously recombinant cells produce an mRNA precursor which includes the non-coding exon introduced upstream of the TPO gene by homologous recombination, the second targeting sequence and any sequences between the second targeting sequence and exon 2 of the TPO gene, and the remaining exons, introns, and 3′ untranslated regions of the TPO gene (FIG. 6). Splicing of this message results in the fusion of the exogenous non-coding exon to exon 2 of the endogenous TPO gene which, when translated, will produce TPO. In this strategy the first and second targeting sequences are upstream of the normal target gene, but this is not required (see below). The size of the intron in the targeting construct and thus the position of the regulatory region relative to the coding region of the gene may be varied to optimize the function of the regulatory region.

Plasmid pRTPO1 is constructed as follows: Based on the restriction map of the TPO upstream region (FIG. 3), a 3.5 kb BamHI fragment can be isolated from subclone pBS(X)/5′Thromb.8 (Example 1). This fragment is ligated to BamHI digested plasmid pBS (Stratagene, Inc., La Jolla, Calif.) and transformed into competent E. coli cells to generate pBS-TPO1. This fragment includes sequences lying upstream of TPO exon 1. Next, a 0.73 kb fragment was amplified from hGH expression construct pXGH308, which has the CMV immediate-early (IE) gene promoter region beginning at nucleotide 546 and ending at nucleotide 2105 of Genbank sequence HS5MIEP fused to the hGH sequences beginning at nucleotide 5225 and ending at nucleotide 7322 of Genbank sequence HUMGHCSA, using oligonucleotides 2.1 and 2.2. (The source of the CMV IE gene is not critical, and other CMV IE promoter-based plasmids may be used, or wild-type CMV DNA may be used.) Oligo 2.1 (37 bp, SEQ ID NO: 5), hybridizes to the CMV IE promoter at −614 relative to the cap site (in Genbank sequence HEHCMVP1), and includes a NotI site followed by a partially overlapping XhoI site at its 5′ end. Oligo 2.2 (36 bp, SEQ ID NO: 6), hybridizes to the CMV IE promoter at +131 relative to the cap site and includes the first 10 base pairs of the first intron of the CMV IE gene and contains a NotI site at its 5′ end. The resulting PCR fragment is digested with NotI and gel-purified. Plasmid pBS-TPO1 is digested with NotI, which cleaves at a single site upstream of TPO exon 1 (FIG. 3), and the digested DNA is ligated to the CMV promoter fragment prepared above and transformed into competent E. coli cells. Colonies containing inserts of the CMV promoter inserted at the NotI site of pBS-TPO1 are analyzed by restriction enzyme analysis to confirm the orientation of the insert, and one recombinant plasmid in which the CMV promoter is oriented such that the direction of transcription is towards TPO exon 1 is identified and designated pBS-TPO2.

Oligo 2.1

(SEQ ID NO: 5)

5′ TTTTGCGGCC GCTCGAGGAC ATTGATTATT GACTAGT
NotI XhoI
Oligo 2.2

(SEQ ID NO: 6)

5′ TTTTGCGGCC GCCGGTACTT ACGTCACTCT TGGCAC
NotI

Next, the neomycin phosphotransferase (neo) gene is inserted into pBS-TPO2 for use as a selectable marker in isolating stably transfected human cells. Plasmid pMC1neoPolyA [Thomas, K. R. and Capecchi, M. R. Cell 51:503-512 (1987); available from Stratagene Inc., La Jolla, Calif.] is digested with BamHI and made blunt-ended by treatment with the Klenow fragment of E. coli DNA polymerase. The treated DNA is then ligated to a double-stranded 10 base pair ClaI linker of the sequence 5′GGATCGATCC, chosen such that the BamHI site is not regenerated by the linker addition. The resulting DNA is digested with ClaI and the digested DNA is ligated under dilute conditions to promote recircularization and transformed into competent E. coli cells. Transformed colonies are analyzed by restriction enzyme digestion to identify cells containing a derivative of plasmid pMC1neoPolyA with an insertion of a ClaI site at the 3′ end of the neo gene. This plasmid is designated pMC1neo-C. pMC1neo-C is digested with XhoI and SalI and the approximately 1.1 kb fragment containing the neo expression unit is gel purified. Plasmid pBS-TPO2 is digested at the unique XhoI site which was introduced by PCR at the 5′ end of the CMV promoter, and the digested DNA is ligated to the purified XhoI-SalI fragment containing the neo gene and transformed into competent E. coli cells. Colonies containing inserts of the neo gene inserted at the XhoI site of pBS-TPO2 are analyzed by restriction enzyme analysis to confirm the orientation of the insert, and one recombinant plasmid in which the neo gene is oriented such that the direction of transcription is opposite to CMV is identified and designated pBS-TPO3.

Finally, the targeting construct pTPO1 is constructed by insertion of a dhfr expression unit (to select for amplification in targeted human cells) at the ClaI site located at the 5′ end of the neo gene of pBS-TPO3. To obtain a dhfr expression unit, the plasmid construct pF8CIS9080 [Eaton et al., Biochemistry 25: 8343-8347 (1986)] is digested with EcoRI and SalI. A 2 kb fragment containing the dhfr expression unit is purified from this digest and made blunt by treatment with the Klenow fragment of DNA polymerase I. A ClaI linker (New England Biolabs, Beverly, Mass.) is then ligated to the blunted dhfr fragment. The products of this ligation are digested with ClaI ligated to ClaI digested pBS-TPO3. An aliquot of this ligation is transformed into E. coli and plated on ampicillin selection plates. Bacterial colonies are analyzed by restriction enzyme digestion to determine the orientation of the inserted dhfr fragment. One plasmid with dhfr in a transcriptional orientation opposite that of the neo gene is designated pRTPO1. For targeting to the TPO locus in cultured human cells, pRTPO1 is digested with BamHI to separate the targeting fragment containing the targeting DNA, neo gene, dhfr gene, CMV promoter, and splice-donor site from the pBS plasmid backbone.

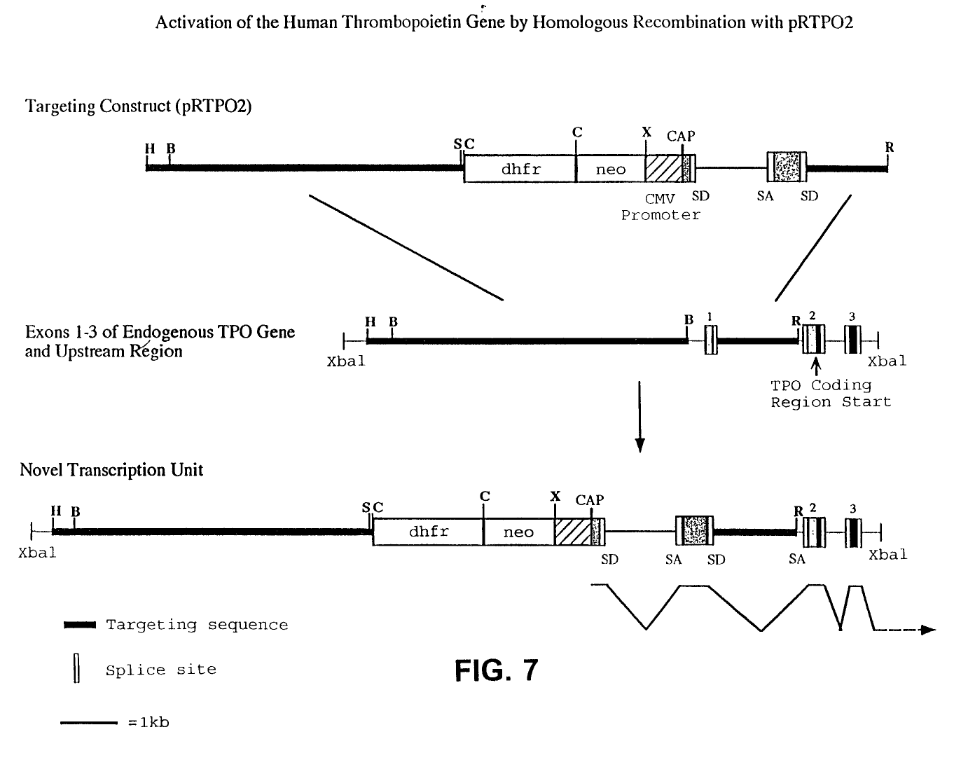

A second strategy for activation of the TPO gene is shown in FIG. 7. In this strategy, a targeting fragment is introduced into the genome of recipient cells for insertion of a regulatory region, a non-coding exon, a splice-donor site, an intron, a splice-acceptor site, a second non-coding exon, and a functional, unpaired splice-donor site upstream of the TPO coding region. Specifically, the targeting construct from which this fragment is derived (pRTPO2) is designed to include a first targeting sequence homologous to sequences upstream of the TPO gene, an amplifiable marker gene, a selectable marker gene, a regulatory region, a CAP site, a non-coding exon, a splice-donor site, an intron, a splice-acceptor site, a second non-coding exon, an unpaired splice-donor site, and a second targeting sequence corresponding to sequences downstream of the first targeting sequence but upstream of TPO exon 2. By this strategy, homologously recombinant cells produce an mRNA precursor which corresponds to the first and second non-coding exogenous exons separated by an intron, the second targeting sequence, any sequences between the second targeting sequence and exon 2 of the TPO gene, and the remaining exons, introns, and 3′ untranslated regions of the TPO gene (FIG. 7). Splicing of this message results in the fusion of the second non-coding exogenous exon to exon 2 of the endogenous TPO gene which, when translated, will produce TPO. In this strategy the first and second targeting sequences are upstream of the normal target gene, but this is not required (see below). The size of the intron in the targeting construct and thus the position of the regulatory region relative to the coding region of the gene may be varied to optimize the function of the regulatory region.

Plasmid pRTPO2 is constructed as follows: Based on the restriction map of the TPO upstream region (FIG. 3), a 1.8 kb BamHI-EcoRI fragment can be. isolated from subclone pBS(X)/5′Thromb.8 (Example 1). This fragment is ligated to BamHI and EcoRI digested plasmid pBS (Stratagene, Inc., La Jolla, Calif.) and transformed into competent E. coli cells to generate pBS-TPO4. This fragment includes TPO exon 1 but contains no TPO coding sequences.

Next, oligonucleotides 2.3 to 2.6 are used in PCR to fuse CMV IE promoter sequences beginning at nucleotlde 546 and ending at nucleotide 2105 of Genbank sequence HS5MIEP to sequences from the TPO gene comprised of exon 1 and a portion of intron 1. The properties of these primers are as follows: 2.3 (SEQ ID NO: 7) is a 30 base oligonucleotide homologous to a segment of the CMV IE promoter beginning at nucleotide 546 of Genbank sequence HS5MIEP (−614 relative to the cap site) and includes a XhoI site at its 5′ end; 2.4 (SEQ ID NO: 8) and 2.5 (SEQ ID NO: 9) are 60 nucleotide complementary primers which define the fusion of CMV (position 2100 of Genbank sequence HS5MIEP) and TPO (position −1881 relative to the TPO translation start site) sequences; 2.6 (SEQ ID NO: 10) is 27 nucleotides in length and is homologous to TPO sequences ending in TPO intron 1 at position −1374 relative to the TPO translation start site and includes a natural ApaI site.

Oligo 2.3

(SEQ ID NO: 7)

5′ TTTTCTCGAG GACATTGATT ATTGACTAGT
XhoI
Oligo 2.4

(SEQ ID NO: 8)

5′ catgggtctt ttctgcagtc accgtccttg CTACCCATCT GCT
CCCCAGA GGGCTGCCTG
Oligo 2.5

(SEQ ID NO: 9)

5′ CAGGCAGCCC TCTGGGGAGC AGATGGGTAG caaggacggt gac
tgcagaa aagacccatg
Oligo 2.6

(SEQ ID NO: 10)

5′ TTTTGGGCCC TCCTCCCATT ACCCTCT
ApaI

Oligos 2.3-2.6: Bases in lower-case type denote CMV sequences; bases in upper-case type denote TPO sequences

These primers are used to amplify a 2.1 kb DNA fragment comprising a fusion of CMV IE and TPO sequences. The fusion fragment is created by first using oligos 2.3 and 2.4 to amplify a 1.6 kb fragment from hGH expression construct pXGH308, which has the CMV immediate-early (IE) gene promoter region beginning at nucleotide 546 and ending at nucleotide 2105 of Genbank sequence HS5MIEP fused to the hGH sequences beginning at nucleotide 5225 and ending at nucleotide 7322 of Genbank sequence HUMGHCSA. (The source of the CMV IE gene is not critical, and other CMV IE promoter-based plasmids may be used, or wild-type CMV DNA may be used.) Then, oligos 2.5 and 2.6 are used to amplify a 0.54 kb fragment containing portions of TPO exon 1 and TPO intron 1 from plasmid pBS(X)/5′Thromb.8 (Example 1). The two amplified fragments are then combined and further amplified using oligos 2.3 and 2.6. The resulting product, a 2.1 kb PCR fragment is digested with XhoI and ApaI and gel purified. Plasmid pMCneo-C (see above) is digested with SalI and XhoI and the 1.1 kb neo containing fragment is gel purified. The purified 2.1 kb PCR fragment and the 1.1 kb neo fragment are then mixed and ligated to pBS-TPO4 (above) which has been cut with SalI and ApaI. The ligation mixture is transformed into E. coli cells and a plasmid with a single insert of each the fusion fragment and the neo gene is identified, this plasmid having the SalI site at the 3′ end of the neo gene regenerated by ligation to the SalI site in the polylinker of pBS-TPO4. The resulting plasmid is designated pBS-TPO5.

A dhfr expression unit (to select for amplification in targeted human cells) is then inserted at the ClaI site located at the 5′ end of the neo gene of pBS-TPO5. The dhfr expression unit is isolated from plasmid pF8CIS9080 [Eaton et al., Biochemistry 25: 8343-8347 (1986)] by digestion with EcoRI and SalI. A 2 kb fragment containing the dhfr expression unit is purified from this digest and made blunt by treatment with the Klenow fragment of DNA polymerase I. A ClaI linker (New England Biolabs, Beverly, Mass.) is then ligated to the blunted dhfr fragment. The products of this ligation are digested with ClaI ligated to ClaI digested pBS-TPO5. An aliquot of this ligation is transformed into E. coli and plated on ampicillin selection plates. Bacterial colonies are analyzed by restriction enzyme digestion to determine the orientation of the inserted dhfr fragment. One plasmid with dhfr in a transcriptional orientation opposite that of the neo gene is designated pBS-TPO6.

To complete plasmid pRTPO2, plasmid pBS(X)/5′Thromb.8 (Example 1) is partially digested with BamHI and ligated to a SalI linker. The resulting DNA is then digested with SalI and HindIII and the 3.7 kb fragment consisting of sequences upstream of the TPO gene is isolated for use as a second targeting sequence. This fragment is ligated to HindIII-SalI digested pBS-TPO6 to generate the targeting plasmid pRTPO2. For targeting to the TPO locus in cultured human cells, pRTPO2 is digested with HindIII and EcoRI to separate the targeting fragment containing the targeting DNA, neo gene, dhfr gene, and CMV promoter from the pBS plasmid backbone.

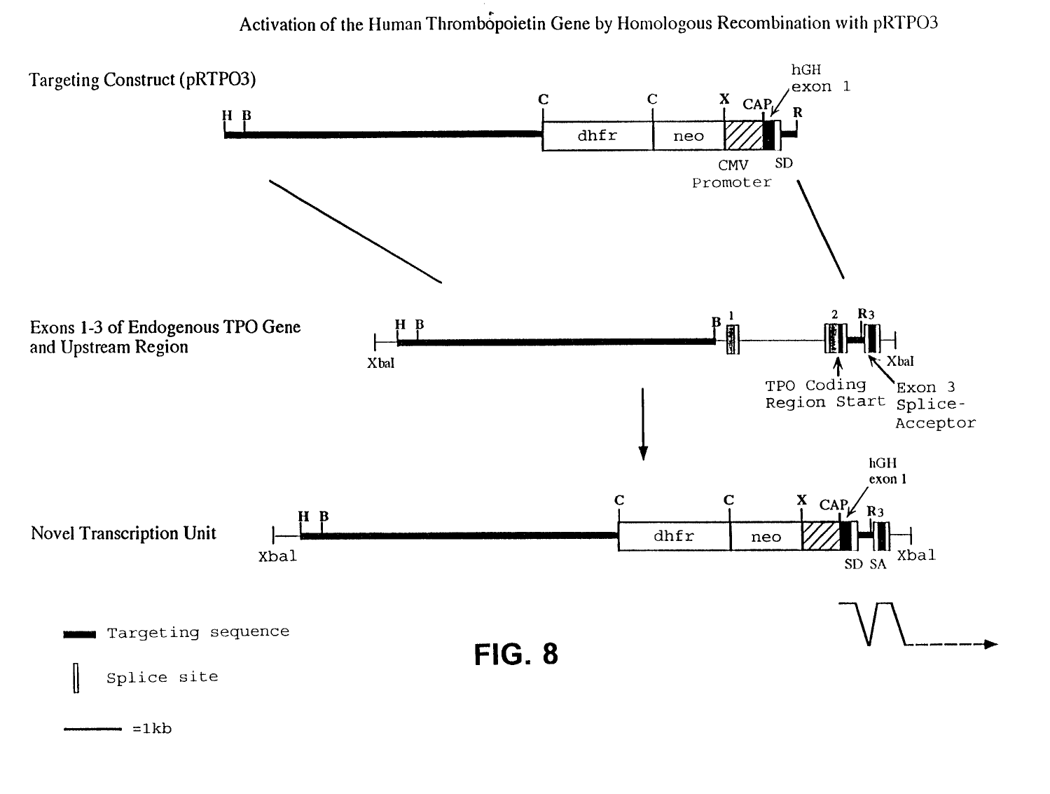

A third strategy for activation of the TPO gene is shown in FIG. 8. In this strategy, a targeting fragment is introduced into the genome of recipient cells for replacement of the normal TPO regulatory region, TPO exon 1, TPO intron 1, and TPO exon 2 with an exogenous regulatory region, a coding exon, and a functional, unpaired splice-donor site. Specifically, the targeting construct from which this fragment is derived (pRTPO3) is designed to include a first targeting sequence homologous to sequences upstream of the TPO gene, an amplifiable marker gene, a selectable marker gene, a regulatory region, a CAP site, an exon which includes sequences coding for the first 3⅓ amino acids of the human growth hormone (hGH) signal peptide, an unpaired splice-donor site, and a second targeting sequence corresponding to TPO intron 2 sequences. By this strategy, homologously recombinant cells produce an mRNA precursor which corresponds to the exogenous coding exon, intron 2 of the TPO gene, exon 3 of the TPO gene, and the remaining exons, introns, and 3′ untranslated regions of the TPO gene (FIG. 8). Splicing of this message results in the fusion of the exogenous coding exon to exon 3 of the endogenous TPO gene which, when translated, will produce a fusion protein in which the first 3 amino acids of the signal peptide are derived from hGH. The signal peptide of this molecule is cleaved off prior to secretion from a cell to produce mature TPO. In this strategy the first targeting sequence is upstream of the normal target gene, while the second targeting sequence is within the gene, between exons 2 and 3. The position of the first targeting sequence and the amount of upstream DNA replaced or deleted by the targeting event may be varied to optimize the function of the regulatory region.

Plasmid pRTPO3 is constructed as follows: oligonucleotides 2.8 to 2.11 are used in PCR to fuse CMV IE promoter sequences beginning at nucleotide 546 and ending at nucleotide 1258 of Genbank sequence HS5MIEP to sequences from the human growth hormone gene which encode the first 3⅓ amino acids of the hGH signal peptide, a splice donor site, and the second intron of the TPO gene. The properties of these primers are as follows: Oligo 2.8 (SEQ ID NO: 11) is a 30 base oligonucleotide homologous to a segment of the CMV IE promoter beginning at nucleotide 546 of Genbank sequence HS5MIEP (−614 relative to the cap site) and includes an XhoI site at its 5′ end; 2.9 (SEQ ID NO: 12) and 2.10 (SEQ ID NO: 13) are 69 nucleotide complementary primers which define the fusion of CMV (position 2100 of Genbank sequence HS5MIEP) and hGH sequences (position −10 relative to the translation start site of the hGH gene; see the hGH gene N sequence in Genbank entry HUMGHCSA) sequences. These primers also include the first 29 base pairs of TPO intron 2 (nucleotides +14 to +42 relative to the TPO translation start site), which include the splice donor site; 2.11 (SEQ ID NO: 14) is 45 nucleotides in length and is homologous to TPO sequences in TPO intron 2 starting at position +182 relative to the TPO translation start site and extending upstream, and includes a natural EcoRI site at its 5′ end.

The fusion fragment is created by first using oligos 2.8 and 2.9 to amplify a 0.7 kb fragment from CMV viral DNA containing a wild-type immediate early gene and promoter sequence. (The source of the CMV IE gene is not critical, and other CMV IE promoter-based plasmids may be used.) Then, oligos 2.10 and 2.11 are used to amplify a 0.17 kb fragment containing a portion of TPO intron 2 from plasmid pBS(X)/5′Thromb.8 (Example 1). The two amplified fragments are then combined and further amplified using oligos 2.8 and 2.11. The resulting product, a 0.9 kb PCR fragment is digested with XhoI and EcoRI and gel purified. Next, plasmid a pBS(X)/5′Thromb.8 (Example 1) is partially digested with BamHI and ligated to an XhoI linker. The resulting DNA is then digested with XhoI and HindIII and the 3.9 kb fragment consisting of sequences upstream of the TPO gene is isolated for use as a second targeting sequence. This fragment contains sequences from −5985 to −2095 relative to the TPO translation start site (FIG. 3). The isolated fragment is then ligated in a mixture containing the 0.9 kb fusion fragment purified above and HindIII and EcoRI digested plasmid pBS (Stratagene, Inc., La Jolla, Calif.) and transformed into competent E. coli cells to generate pBS-TPO7.

For insertion of the neo selectable marker gene, plasmid pMC1neo-C (see above) is digested with XhoI and SalI and ligated to XhoI digested pBS-TPO7. The ligation mix is transformed into E. coli cells and colonies are analyzed by restriction enzyme analysis to identify a plasmid with a single insert of the neo gene oriented such that the direction of transcription is opposite to that of the CMV promoter. This plasmid is designated pBS-TPO8.

A dhfr expression unit (to select for amplification in targeted human cells) is then inserted at the ClaI site located at the 5′ end of the neo gene of pBS-TPO8. The dhfr expression unit is isolated from plasmid pF8CIS9080 [Eaton et al., Biochemistry 25: 8343-8347 (1986)] by digestion with EcoRI and SalI. A 2 kb fragment containing the dhfr expression unit is purified from this digest and made blunt by treatment with the Klenow fragment of DNA polymerase I. A ClaI linker (New England Biolabs, Beverly, Mass.) is then ligated to the blunted dhfr fragment. The products of this ligation are digested with ClaI ligated to ClaI digested pBS-TPO8. An aliquot of this ligation is transformed into E. coli and plated on ampicillin selection plates. Bacterial colonies are analyzed by restriction enzyme digestion to determine the orientation of the inserted dhfr fragment. One plasmid with dhfr in a transcriptional orientation opposite that of the neo gene is designated pRTPO3. For targeting to the TPO locus in cultured human cells, pRTPO3 is digested with EcoRI and HindIII to separate the targeting fragment containing the targeting DNA, neo gene, dhfr gene, CMV promoter, and hGH coding DNA from the pBS plasmid backbone.

Oligo 2.8

(SEQ ID NO: 11)

5′ TTTTCTCGAG GACATTGATT ATTGACTAGT
XhoI
Oligo 2.9

(SEQ ID NO: 12)

5′ cgcggattcc ccgtgccaag CCTAGCGGCA ATGGCTACAG GTG
AGAACAC ACCTGAGGGG CTAGGGCCA
Oligo 2.10

(SEQ ID NO: 13)

5′ TGGCCCTAGC CCCTCAGGTG TGTTCTCACC TGTAGCCATT GCC
GCTAGGcttggcacggg gaatccgcg
Oligo 2.11

(SEQ ID NO: 14)

5′ TTTTGAATTC CCATTCAGGA COCAGACCTG AAACCCAGGG AAT
EcoRI
CC

Oligos 2.8-2.11: Bases in lower-case type denote CMV sequences; upper-case, non-bold bases denote TPO sequences; boldface bases denote hGH exon 1 sequences.

Other approaches for targeting and activation of the TPO gene may be employed. For example, the first and second targeting sequences may correspond to sequences in the first or second intron of the TPO gene, and the targeting sequences may include TPO coding sequences. In any activation strategy, the second targeting sequence does not need to lie immediately adjacent to or near the first targeting sequence in the normal gene, such that portions of the gene's normal upstream region are deleted upon homologous recombination. Furthermore, one targeting sequence may be upstream of the gene and one may be within an exon or intron of the TPO gene.

A selectable marker gene is optional and the amplifiable marker gene is only required when amplification is desired. The amplifiable marker gene and selectable marker gene may be the same gene, their positions may be reversed, and one or both may be situated in the intron of the targeting construct. Amplifiable marker genes and selectable marker genes suitable for selection are described herein. The incorporation of a specific CAP site is optional. The regulatory region, CAP site, first non-coding exon, splice-donor site, intron, second non-coding exon, and splice acceptor site may be isolated as a complete unit from the human elongation factor-la (EF-1a; Genbank sequence HUMEF1A) gene or the cytomegalovirus (CMV; Genbank sequence HEHCMVP1) immediate early region, or the components can be assembled from appropriate components isolated from different genes. In any case, either exogenous exon may be the same or different from the first exon of the normal TPO gene, and multiple non-coding exons may be present in the targeting construct.

As described herein, a number of selectable and amplifiable markers may be used in the targeting constructs, and the activation may be effected in a large number of cell-types.

### EXAMPLE 3

### In vitro Production of TPO by Activation and Amplification of the TPO Gene in an Immortalized Cell Line

Transfection of primary, secondary, or immortalized human cells and isolation of homologously recombinant cells expressing TPO may be accomplished using the methods described in U.S. Ser. No. 08/243,391 incorporated by reference. Homologously recombinant cells may be identified by PCR screening strategy as exemplified therein and in published methods available to one skilled in the art (see, for example, Kim, H-S and Smithies, O., Nucl. Acids Res. 16:8887-8903 (1988)). The identification of cells expressing TPO may also be accomplished using a variety of assays based on the structure or properties of TPO. For example, TPO may be functionally identified by an in vitro or in vivo megakaryocytopoiesis assay (de Sauvage et al., Nature 369:533-538 (1994)). Alternatively, TPO may be assayed by the stimulation of proliferation of cells expressing the c-mpl ligand, the receptor for TPO. In this assay, cells such as Ba/F3-mpl cells (de Sauvage et al., Nature 369:533-538 (1994)), are exposed to TPO and cell proliferation is monitored by ³H-thymidine uptake. TPO may also be assayed through its effects on in vivo platelet production, either by direct platelet counts or by incorporation of ³⁵S into platelets. Finally, peptides corresponding to portions of the TPO molecule may be synthesized in order to generate anti-TPO antibodies for use in an ELISA assay.

The isolation of cells containing amplified copies of the amplifiable marker gene and the activated TPO locus is performed as described in U.S. Ser. No. 07/985,586 incorporated by reference.

### EXAMPLE 4

### Cloning of the Human DNase I Gene and Identification of the 5′Flanking Sequences

The human DNase I gene was isolated from a human genomic DNA library. The library (Clontech, Palo Alto, Calif.; Cat. #HL1006d) was constructed by cloning MboI partially digested male leukocyte DNA into the BamHI site of the bacteriophage lambda vector EMBL3. For library screening, a DNA probe was isolated by PCR amplification of human genomic DNA using oligonucleotides 4.1 and 4.2.

Oligo 4.1
(SEQ ID NO: 15)
5′ TGCCTTGAAG TGCTTCTTCA
Oligo 4.2
(SEQ ID NO: 16)

5′ CCTCAGAGAT GACGAGAATG C

These primers were designed based on the published DNase I mRNA sequence (Shak S. et al., Proc. Natl. Acad.

Sci. USA 87:9188-9192 (1990)). The amplified probe (probe A; 126 bp) was labeled with ³²P-dCTP by PCR and used to screen a bacteriophage lambda genomic DNA library. The filters were hybridized for 16 hours at 68° C. in 125 mM Na₂HPO₄ (pH 7.2), 250 mM NaCl, 10% PEG 8000, 7% SDS, 1 mM EDTA. Filters were washed two times in 500 ml of 20 mM Na₂HPO₄ (pH 7.2), 5% SDS, 1 mM EDTA, followed by 4 washes in 500 ml of 20 mM Na₂HPO₄ (pH 7.2), 1% SDS, 1 mM EDTA.

The wash buffers were preheated to 56° C. and washing was performed at room temperature on a rotary shaker for approximately 5 minutes per wash. The hybridization signals were visualized by autoradiography at −80° C. with an intensifying screen. In this experiment, approximately 1×10⁶ phage were screened and 18 positive signals were obtained. Bacteriophage plaques corresponding to 10 of the positive signals were plated at low density and subjected to a second round of screening using probe A. Four of the phage (designated 2a, 3b, 4c and 14a) gave positive hybridization signals following the secondary screening and were retained for further analysis. DNA was isolated from the plaque purified phage following amplification and subsequent purification by cesium chloride gradient ultra centrifugation (Yamamoto, K. R. et al., Virology 40:734 (1970)). Library screening, plaque purification of recombinant bacteriophage and isolation of bacteriophage DNA was performed using standard methods (Ausubel et al., Current Protocols in Molecular Biology. Wiley, New York, N.Y. (1987)).

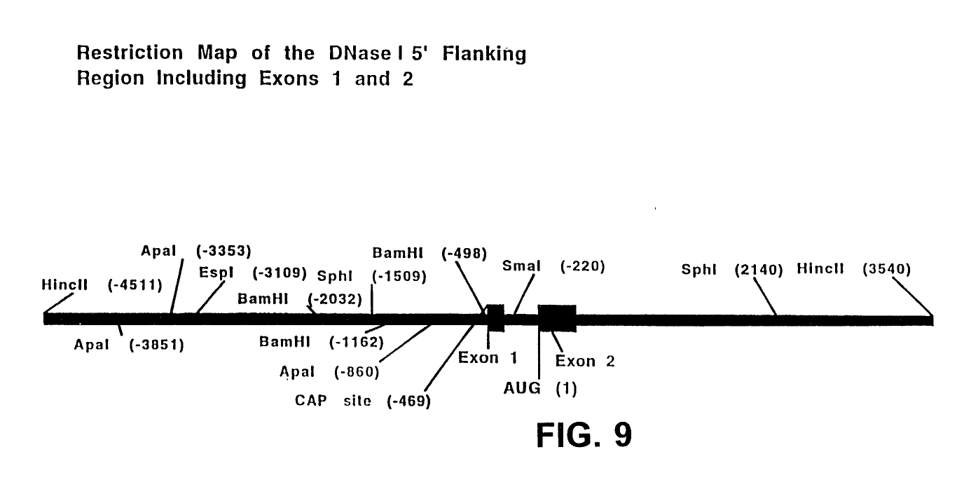

Based on restriction enzyme digestion and Southern blot analysis using probe A, two of the phage (4c and 14a) contain a common HincII fragment of approximately 8 kb which encompasses exon 1, intron 1, exon 2, coding and non-coding sequences corresponding to intron 2 and downstream DNase I exons, as well as approximately 4 kb of non-transcribed DNA lying upstream of DNase I exon I. This fragment was isolated from one genomic clone (4c) and subcloned into pBSIISK⁺ (Stratagene Inc., La Jolla, Calif.) for further analysis. Restriction enzyme mapping of the resultant clone, pBS/ 4C.2Hinc2, was used to generate the restriction map shown in FIG. 9. The nucleotlde sequence of the non-transcribed DNase I 5′ region lying upstream of the 5′ end of the known cDNA sequence is shown in FIG. 10 (SEQ ID NO: 17). The nucleotide sequence lying downstream of the 5′ end of the known cDNA sequence, including exon 1, intron 1 and part of exon 2 is shown in FIG. 11 (SEQ ID NO: 18). Comparison of the cloned genomic sequence presented here, with the published cDNA sequence (Shak, S. et al., Proc. Natl. Acad. Sci. USA 87:9188-9192 (1990)) reveals that the 5′ end of the DNase I gene consists of a non-coding exon (exon 1) of 142 bp and a second exon (exon 2) which is at least 341 bp. Exon 2 encodes a 22 amino acid signal sequence and a portion of the mature DNase I peptide, beginning with an AUG translational initiation codon which lies 1 bp downstream of the 5′ end of exon 2. Exons 1 and 2 are separated by intron 1 which is 336 bp in length.

### EXAMPLE 5

### Construction of Targetinq Plasmids for Activation and Amplification of the DNase I Gene

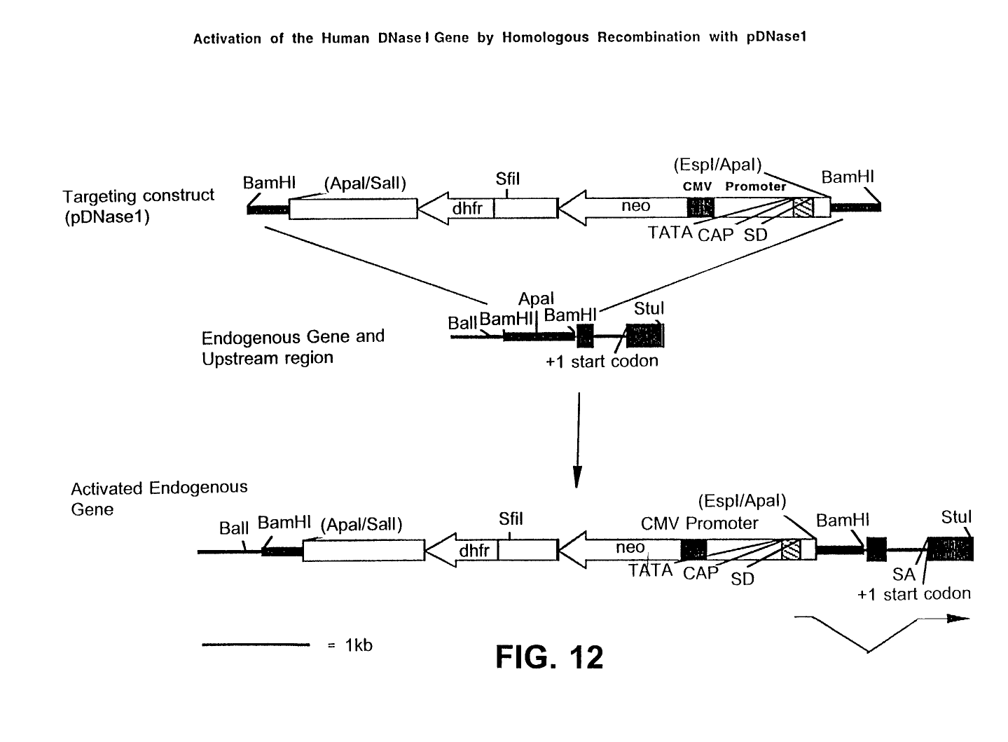

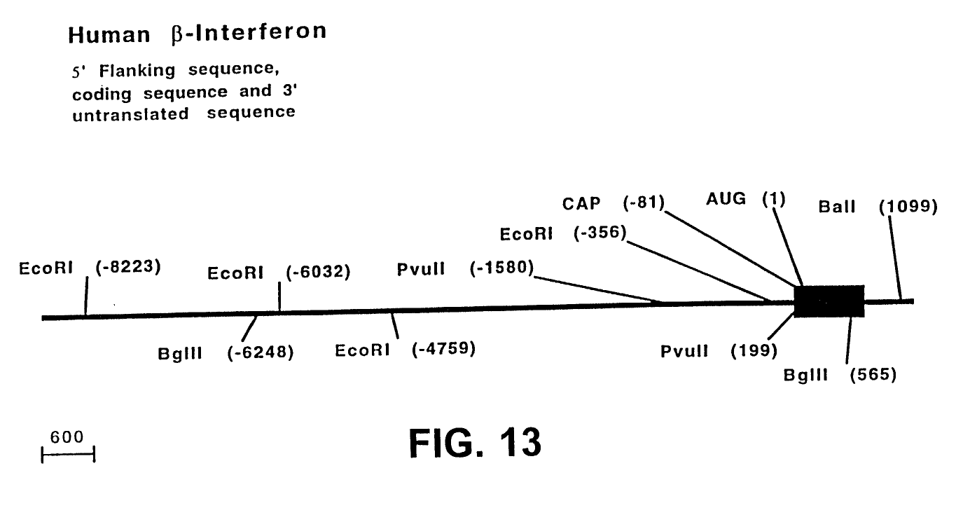

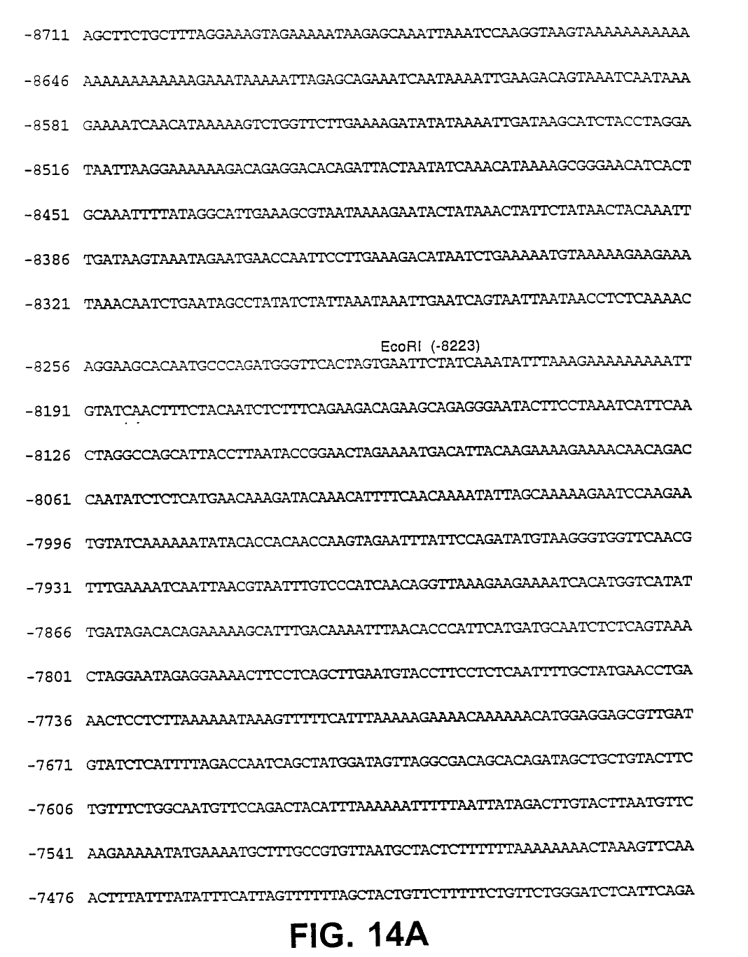

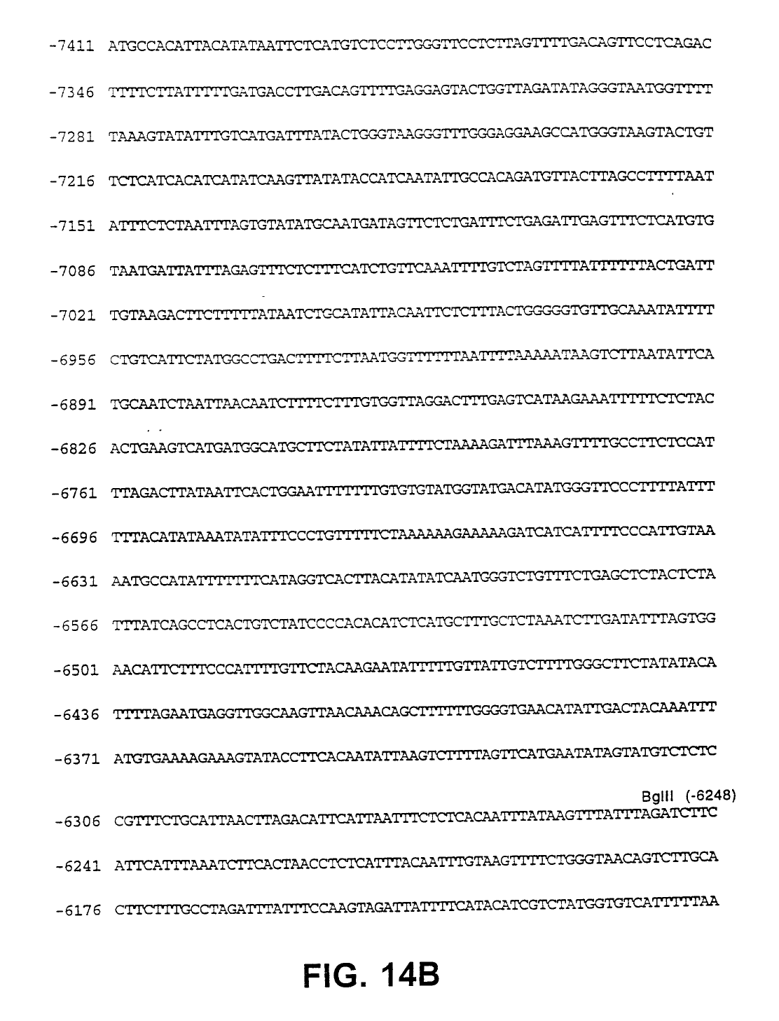

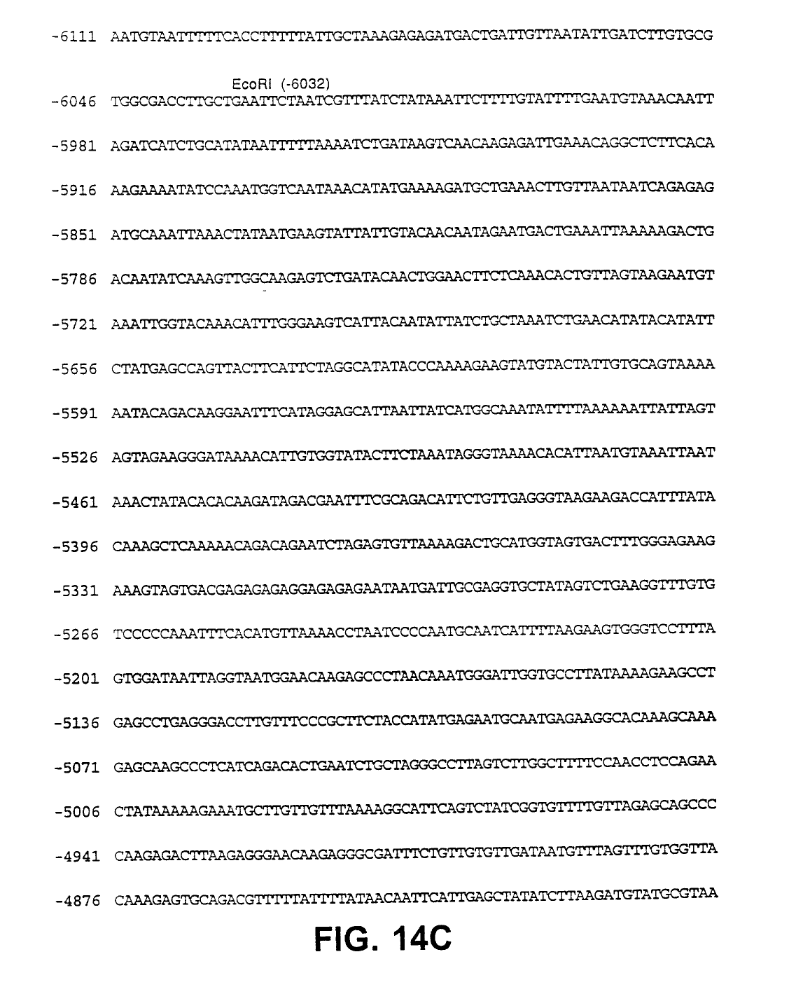

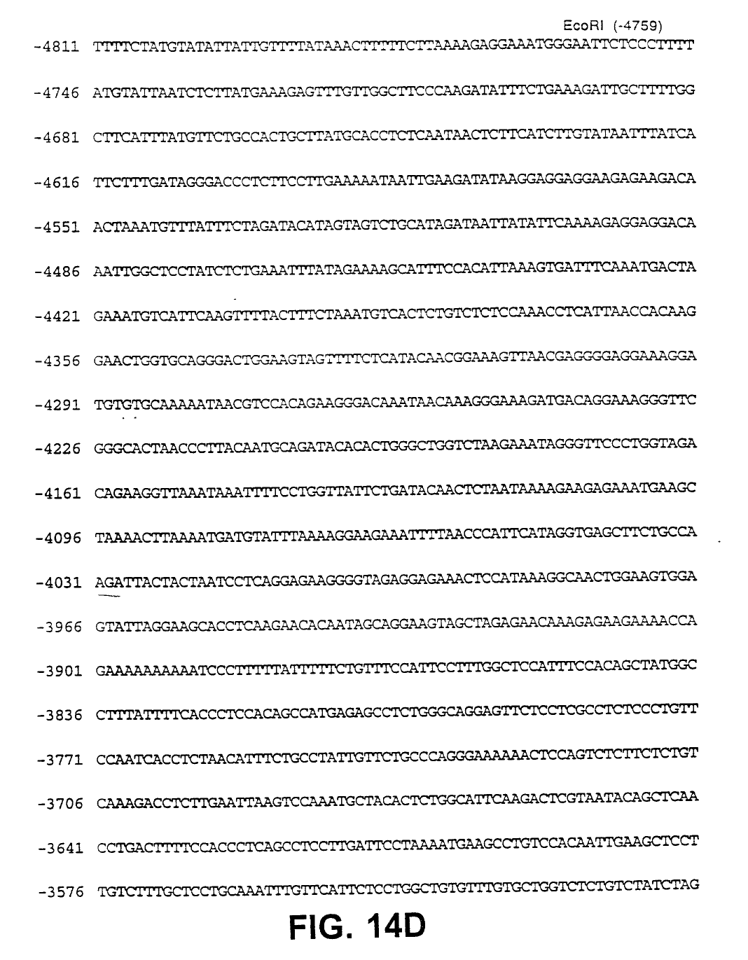

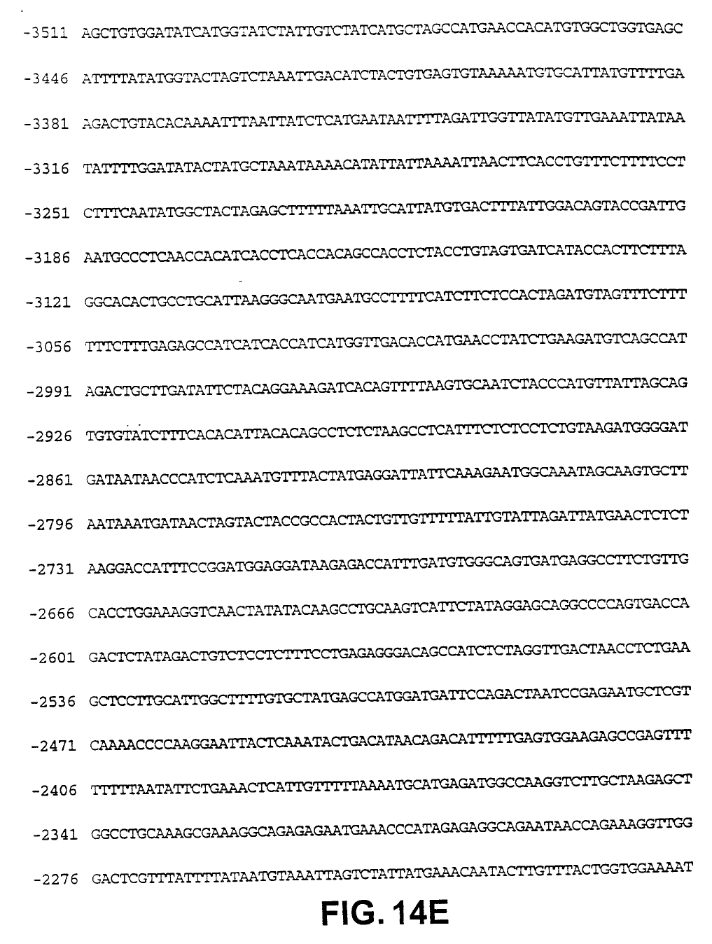

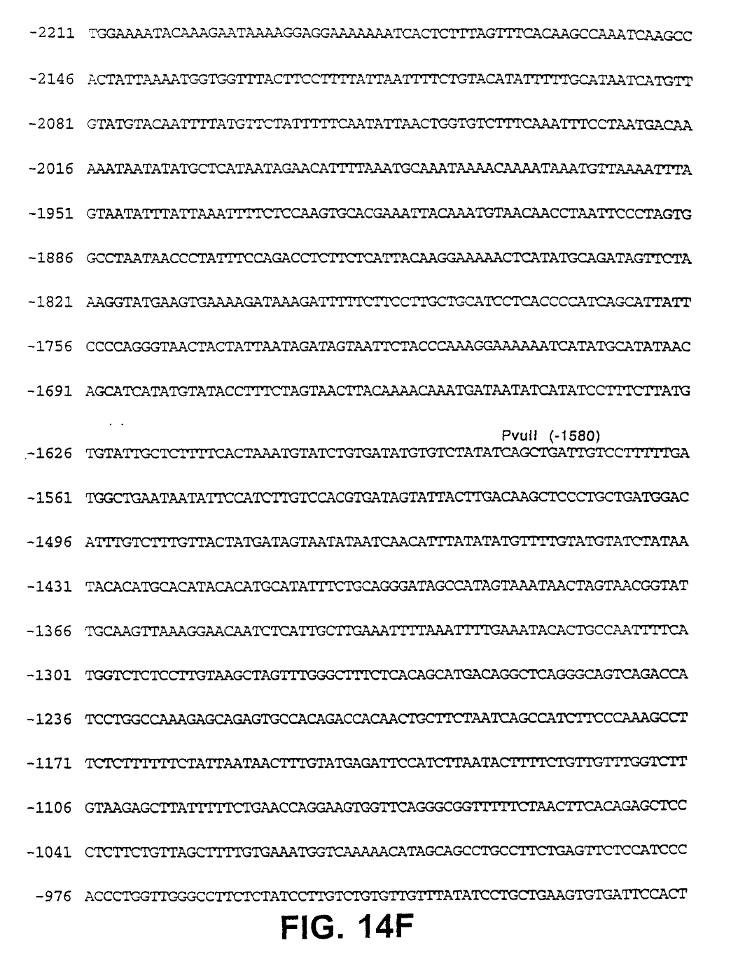

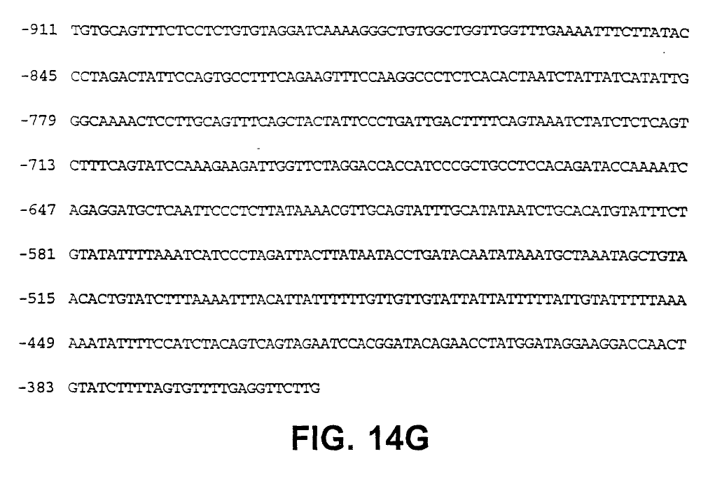

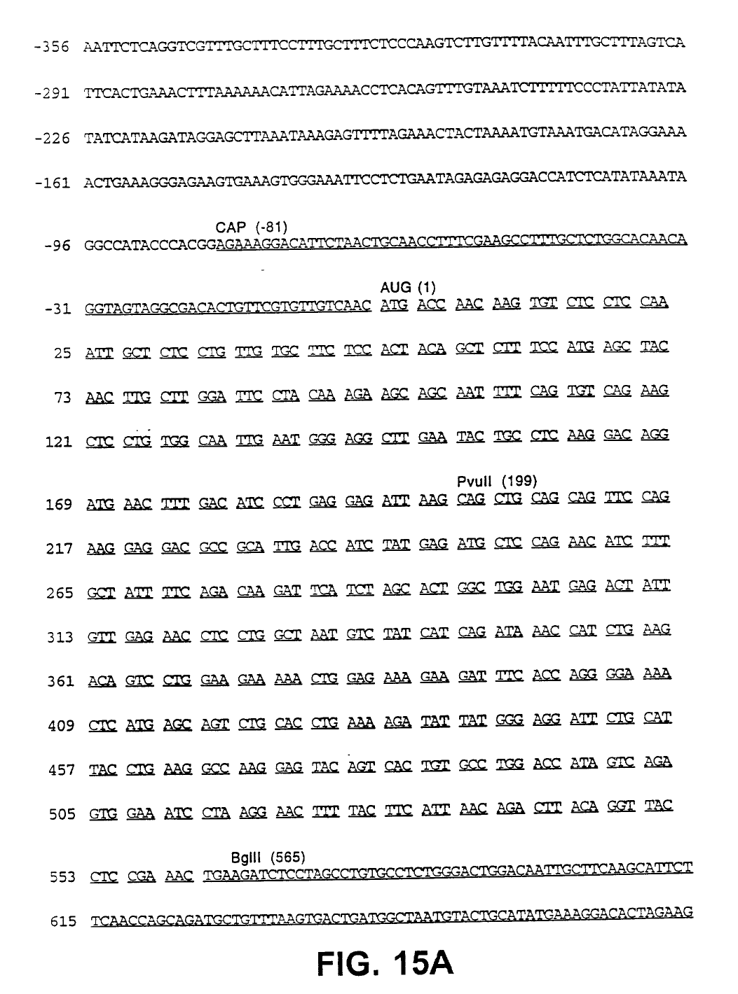

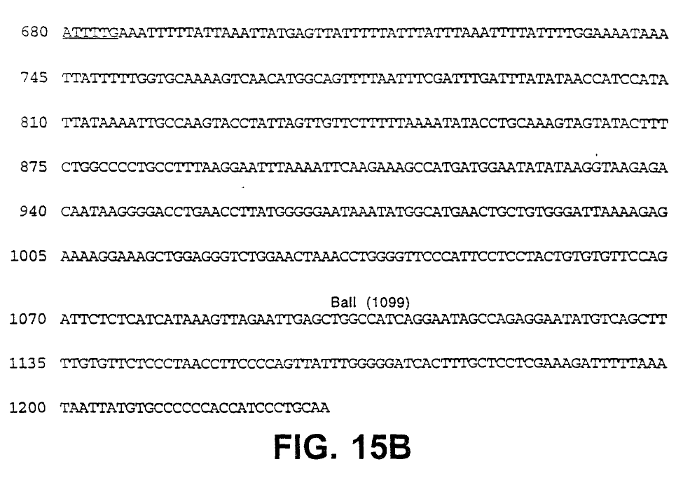

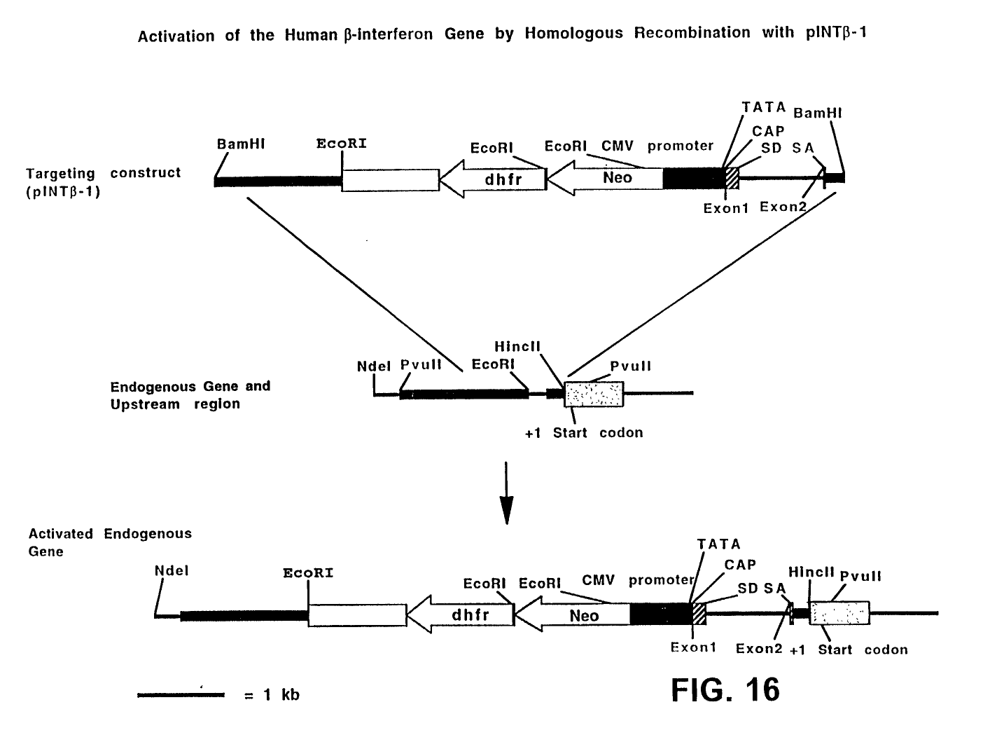

The activation of the DNase I gene can be accomplished by the strategy outlined in FIG. 12. In this strategy, a targeting fragment is introduced into the genome of recipient cells for insertion of a regulatory region, a non-coding exon and a functional unpaired splice-donor site upstream of the DNase I coding region. Specifically, the targeting construct from which this fragment is derived (pDNase1), is designed to include a 5′ targeting sequence homologous to sequences upstream of the DNase I gene, a selectable marker gene, an amplifiable marker gene, a regulatory region, a CAP site, a non-coding exon, an unpaired splice-donor site, and a 3′ targeting sequence corresponding to sequences downstream of the 5′ targeting sequence but upstream of DNase I exon 1. According to this strategy, integration of the targeting construct by homologous recombination generates recombinant cells producing an mRNA precursor which includes the non-coding exon introduced upstream of the DNase I gene, the 3′ targeting sequence, any sequences between the 3′ targeting sequence and exon 2 of the DNase I gene, and the remaining exons, introns and 3′ untranslated regions of the DNase I gene (FIG. 12). Splicing of this transcript results in the fusion of the exogenous non-coding exon to exon 2 of the endogenous DNase I gene. DNase I is produced by translation of the mature mRNA. According to this strategy, both the 5′ and 3′ targeting sequences are upstream of the endogenous target gene. The size of the chimeric intron in the targeting construct, which is dictated by the position of the regulatory region relative to the coding sequence, may be varied to optimize the function of the regulatory region.

Plasmid pCND1, which contains the activation cassette, is constructed as follows: A 1555 bp (size includes a 9 bp synthetic HindIII recognition site at the 5′ end of oligo 5.2) fragment is amplified using oligos 5.1 and 5.2. The amplified fragment encompasses the CMV IE promoter, CMV IE exon 1 (non-coding exon) and 827 bp of CMV IE intron 1, beginning at nucleotide 172,783 and ending at nucleotide 174,328 of EMBL sequence X17403 ((Human cytomegalovirus strain AD169). (The source of the CMV IE gene is not critical, and CMV IE promoter-based plasmids or wild-type CMV DNA may be used.) Oligo 5.1 (21 bp, SEQ ID NO: 19) hybridizes to the CMV IE promoter at −598 relative to the CAP site (EMBL sequence X17403). Oligo 5.2 (32 bp, SEQ ID NO: 20) contains 23 nucleotides which hybridize to the CMV IE promoter at +946 relative to the CAP site, the additional 9 bp at the 5′ end of the oligo create a synthetic HindIII recognition sequence. The 1555 bp PCR product is digested with HindIII and the resultant 1551 bp fragment is purified and used in the ligation described below. Next, the neomycin phosphotransferase (neo) gene is isolated from plasmid pBSneo for use as a selectable marker for the isolation of stably transfected human cells. The neo gene in plasmid pBSneo was obtained by BamHI and XhoI digestion of pMC1neo-polyA (Thomas, K. R. and Capecchi, M. R. Cell 51:503-512 (1987)). Plasmid pMC1neo-polyA was digested with BamHI and made blunt ended with the Klenow fragment of E. coli DNA polymerase I. The resulting DNA was digested with XhoI, and the blunt-ended BamHI-XhoI fragment was cloned into HincII and XhoI digested plasmid pBSIISK⁺. For isolation of the neo gene harbored on pBSneo, plasmid pBSneo is digested with XhoI and made blunt-ended by treatment with the Klenow fragment of E. coli DNA polymerase I. The resulting DNA is digested with HindIII and an 1165 bp fragment containing the neo expression unit is gel purified. The 1165 bp neo fragment and the 1551 bp CMV promoter fragment are ligated, the ligation products are digested with HindIII and the 2716 bp HindIII fragment, resulting from blunt-end ligation of the two fragments, is gel purified. The 2716 bp HindIII product is ligated to HindIII digested plasmid pBSIISK⁺ (Stratagene Inc., La Jolla, Calif.) and electroporated into E. coli. Colonies containing inserts in the HindIII site of pBSIISK+are analyzed by restriction enzyme analysis to confirm the orientation of the insert. One recombinant plasmid in which the CMV promoter is oriented such that the oligo 5.2 sequences (+946 relative to the CMV IE CAP site) are proximal to the SalI recognition sequence in the pBSIISK⁺ polylinker, is identified and designated pCN1.

Oligo 5.1
(SEQ ID NO: 19)
5′ GACATTGATT ATTGACTAGT T
Oligo 5.2
(SEQ ID NO: 20)

5′ TTTAAGCTTC TGCAGAAAAG ACCCATGGAA AG

Next, the dhfr expression unit is inserted at a ClaI site which is located at the 3′ end of the neo gene of pCN1. The dhfr expression unit is obtained by EcoRI and SalI digestion of plasmid pF8CIS9080 (Eaton et al., Biochemistry 25:8343-8347 (1986)). The resultant 2 kb fragment is purified from the digest and made blunt with the Klenow fragment of E. coli DNA polymerase I. A ClaI linker (5′CCATCGATGG (NEB 1088; New England Biolabs, Beverly, Mass.) is ligated to the blunt-end dhfr fragment and the ligation products are digested with ClaI. pCN1 is digested with ClaI, and the ClaI dhfr containing fragment is ligated into ClaI site of pCN1. An aliquot of the ligation reaction is electroporated into E. coli and colonies harboring inserts in a ClaI site of pCN1 are analyzed by restriction enzyme analysis to determine the site of insertion and the orientation of the insert. A plasmid with the dhfr expression unit at the 3′ end of the neo gene and with the same transcriptional orientation as that of the neo gene is identified and designated pCND1.

Plasmid pDNase1 is constructed as follows: Based on the restriction map of the upstream region of the DNase I gene (FIG. 9), a 664 bp BamHI fragment (−1161 to −498 in FIG. 8) can be isolated from subclone pBS/4C.2Hinc2. This fragment is ligated to BamHI digested plasmid pBSIISK+dApaI (modification of pBSIISK+; Stratagene Inc., La Jolla, Calif.) in which the ApaI recognition sequence in the polylinker is destroyed. pBSIISK⁺dApaI is constructed by digesting pBSIISK+with ApaI, conversion of the cohesive-ends to blunt-ends with T4 DNA polymerase and ligation to generate the circular plasmid. Following ligation of the 664 bp BamHI fragment into pBSIISK+dApaI, the ligation products are electroporated into E. coli cells to generate pBS-DNase1. The sequences contained in this fragment reside upstream of DNase I exon 1, position −1162 to −498 with respect to the AUG translational initiation codon (nucleotide +1). The activation cassette which contains the CMV immediate-early (IE) promoter region, the CMV IE CAP site, a non-coding exon, an unpaired splice donor site, the neomycin phosphotransferase (neo) selectable marker gene and dhfr expression unit (to select for amplification in targeted human cells) is cloned into the unique ApaI site of the 664 bp BamHI fragment (DNase I upstream region) in pBS-DNase1 (see FIG. 12). Specifically, plasmid pCND1 which contains the activation cassette, is digested with SalI which cuts downstream of the dhfr expression unit and EspI which cuts 242 bp downstream of the CMV IE CAP site. A 3,955 bp SalI-EspI fragment containing the activation cassette is purified from this digest and the cohesive-ends are made blunt by treatment with the Klenow fragment of E. coli DNA polymerase I. This fragment is ligated to plasmid pBS-DNase1, which has been digested with ApaI and made blunt-ended by treatment with T4 DNA polymerase I, and electroporated into E. coli. Colonies containing inserts of the activation cassette inserted at the blunt-ended ApaI site of pBS-DNase 1 are analyzed by restriction enzyme analysis to confirm the orientation of the insert. One recombinant plasmid in which the CMV promoter is oriented such that the direction of transcription is towards DNase I exon 1 is identified and designated pDNase1.

Plasmid pDNase1 is digested with BamHI for transfection into human cells. Transfection of primary, secondary, or immortalized human cells and isolation of homologously recombinant cells expressing DNase I may be accomplished using the methods described in U.S. Ser. No. 08/243,391 and incorporated herein by reference. Homologously recombinant cells may be identified by PCR screening strategy as exemplified therein and in published methods available to one skilled in the art (see, for example, Kim, H-S and Smithies, O., Nucl. Acids Res. 16:8887-8903 (1988)). The identification of cells expressing DNase I may also be accomplished using a variety of assays based on the structure or properties of DNase I. For example, DNase I may be functionally identified by an in vitro enzyme assay (cf. Kunitz, J. Gen. Physiol. 33: 349 (1950); McDonald, Meth. Enzymol. 2:437 (1955)) or by the use of anti-DNase I antibodies in an ELISA assay.

The isolation of cells containing amplified copies of the amplifiable marker gene and the activated DNase I locus is performed as described in U.S. Ser. No. 07/985,586 incorporated herein by reference.

### EXAMPLE 6

### Cloning of the Human β-Interferon Gene and Identification of the 5′Flanking Sequences

The human β-interferon gene was isolated from a human genomic DNA library. The library (Clontech, Palo Alto, Calif.; Cat. #HL1006d) was constructed by cloning MboI partially digested male leukocyte DNA into the BamHI site of the bacteriophage lambda vector EMBL3. For library screening, a DNA probe was isolated by PCR amplification of human genomic DNA using oligonucleotides 6.1 and 6.2

Oligo 6.1
(SEQ ID NO: 21)
5′ TGCTCTGGCA CAACAGGTAG
Oligo 6.2
(SEQ ID NO: 22)

5′ CATAGATGGT CAATGCGGC

These primers were designed based on the published β-interferon mRNA sequence (May, L. T. and Sehgal, P. B., J. Interferon Res. 5:521-526 (1985)). The amplified probe (probe A; 290 bp) was labeled with ³²P-dCTP by PCR and used to screen a bacteriophage lambda genomic DNA library. The filters were hybridized for 16 hours at 68° C. in 125 mM Na₂HPO₄ (pH 7.2), 250 mM NaCl, 10% PEG 8000, 7% SDS, 1 mM EDTA. Filters were washed two times in 500 ml of 20 mM Na₂HPO₄ (pH 7.2), 5% SDS, 1 mM EDTA, followed by 4 washes in 500 ml of 20 mM Na₂HPO₄ (pH 7.2), 1% SDS, 1 mM EDTA. The wash buffers were preheated to 56° C. and washing was performed at room temperature on a rotary shaker for approximately 5 minutes per wash. The hybridization signals were visualized by autoradiography at −80° C. with an intensifying screen. In this experiment, approximately 1×10⁶ phage were screened and 6 positive signals were obtained. Bacteriophage plaques corresponding to the positive signals were plated at low density and subjected to a second round of screening using probe A. Five of the phage (designated 1a, 2a, 2b, 11a, and 12a) gave positive hybridization signals following the secondary screening and were retained for further analysis. DNA was isolated from the plaque purified phage following amplification and subsequent purification by cesium chloride gradient ultra centrifugation (Yamamoto, K. R. et al., Virology 40:734 (1970)). Library screening, plaque purification of recombinant bacteriophage and isolation of bacteriophage DNA was performed using standard methods (Ausubel et al., Current Protocols in Molecular Biology. Wiley, New York, N.Y. (1987)).

Based on restriction enzyme digestion and Southern blot analysis using probe A, all five of the phage (1a, 2a, 2b, 11a, and 12a) were shown to contain a common HindIII fragment of approximately 10 kb which encompasses the entire sequence coding for β-interferon (561 bp), 666 bp of 3′ untranslated sequence and approximately 9 kb of non-transcribed DNA lying upstream of the β-interferon gene. This fragment was isolated from one genomic clone (1a) and subcloned into pBSIISK⁺ (Stratagene Inc., La Jolla, Calif.) for further analysis. The resultant clones, pBS-H3/Bint.11-3 and pBS-H3/Bint.11-21, harbor the 10 kb HindIII fragment in opposite orientations with respect to the plasmid backbone. Restriction enzyme mapping was used to generate the restriction map shown in FIG. 13. The nucleotide sequence of 8,355 bp of DNA lying upstream of the previously reported sequence (Genbank entry HUMIFNB1F) is shown in FIG. 14 (SEQ ID NO: 23). The nucleotide sequence corresponding to 356 bp of DNA upstream of the β-interferon coding region, the β-interferon coding region, and 666 bp of 3′ untranslated sequence is shown in FIG. 15 (SEQ ID NO: 24). Comparison of the cloned genomic sequence presented here, with the published cDNA sequence (May, L. T. and Sehgal, P. B., J. Interferon Res. 5:521-526 (1985)) confirms that the β-interferon gene consists of a 561 bp coding region which is co-linear with its cognate mRNA (lacks introns). The β-interferon gene encodes a 21 amino acid signal sequence and a 120 amino acid mature peptide, beginning with an AUG translational initiation codon which lies 82 bp downstream of the CAP site.

### EXAMPLE 7

### Construction of Targeting Plasmids for Activation and Amplification of the β-Interferon Gene

The activation of the β-interferon gene can be accomplished by the strategy outlined in FIG. 16. In this strategy, a targeting fragment is introduced into the genome of recipient cells for replacement of the endogenous β-interferon regulatory region with an exogenous regulatory region, a non-coding exon, an intron, and chimeric exon sequences consisting of sequences from a noncoding exon (derived from exon 2 of the CMV IE gene) and sequences from the β-interferon 5′ noncoding region. Specifically, the targeting construct from which this fragment is derived (pIFNβ-1) is designed to include a 5′ targeting sequence homologous to sequences upstream of the β-interferon gene, a selectable marker gene, an amplifiable marker gene, a regulatory region, a CAP site, a non-coding exon, an intron, chimeric exon sequences consisting of CMV IE exon 2 sequences and β-interferon 5′ noncoding DNA, and a 3′ targeting sequence homologous to DNA upstream of the β-interferon coding region. According to this strategy, integration of the targeting construct by homologous recombination generates recombinant cells producing an mRNA precursor which includes the non-coding exon introduced upstream of the β-interferon gene, an intron, the chimeric exon which fuses CMV IE exon sequences to β-interferon 5′ noncoding sequences and the entire β-interferon coding region, and 3′ untranslated regions of the β-interferon gene (FIG. 16). The chimeric exon consists of 17 bp of CMV IE exon 2 (position 172,782 to 172,766 of EMBL sequence X17403) joined to the 5′ flanking region of the β-interferon gene (position −173 with respect to the AUG translational initiation codon). Splicing of this transcript results in the fusion of the exogenous non-coding exon to exon 2 which includes the complete coding sequence of the endogenous β-interferon gene. β-interferon is produced by translation of the mature mRNA. According to this strategy, the 5′ targeting sequence is upstream of the endogenous target gene and the 3′ targeting sequence is in the β-interferon 5′ noncoding region. The position of the regulatory region relative to the 5′ flanking sequence, may be varied (e.g. by altering the size of the intron in the targeting construct) to optimize the function of the regulatory region.

Plasmid pIFNβ-1 is constructed as follows: A 182 bp fragment (size includes a 9 bp synthetic BamHI recognition site at the 5′ end of Oligo 7.1) is amplified from pBS-H3/Bint.11-3 using oligos 7.1 and 7.2. The amplified fragment serves as the 3′ targeting sequence (FIG. 16). oligo 7.1 (21 bp, SEQ ID NO: 25) hybridizes to the β-interferon 5′ non-transcribed region at position −173 with respect to the 1-interferon AUG translational initiation codon (FIG. 15). Oligo 7.2 (30 bp, SEQ ID NO: 26) contains 21 nucleotides which hybridize to the β-interferon 5′ untranslated region at position −1 relative to the AUG translational start codon (see FIG. 16), with the additional 9 bp at the 5′ end of the oligo creating a synthetic BamHI recognition sequence. The 182 bp PCR product is purified and used in the ligation described below. Next, a 1571 bp (size includes an 8 bp synthetic SmaI recognition sequence at the 5′ end of oligo 7.3) fragment is amplified using oligos 7.3 and 7.4. The amplified fragment encompasses the CMV IE promoter, CMV IE exon 1 (non-coding exon), CMV IE intron 1 and 17 bp of CMV IE exon 2, beginning at nucleotide 174,328 and ending at nucleotide 172,766 of EMBL sequence X17403 (Human cytomegalovirus strain AD 169). (The source of the CMV IE gene is not critical, and CMV IE promoter-based plasmids or wild type CMV DNA may be used). Oligo 7.3 (29 bp, SEQ ID NO: 27) contains 21 nucleotides which hybridize to the CMV IE promoter at −598 relative to the CAP site (EMBL sequence X17403), the 5′ end of the oligo also contains a 8 bp synthetic SmaI recognition sequence. Oligo 7.4 (21 bp, SEQ ID NO: 28) hybridizes to the CMV IE promoter at +965 relative to the CAP site. The 1571 bp PCR product containing the CMV IE promoter, CMV IE exon 1, CMV IE intron 1 and 23 bp of CMV IE exon 2, is gel purified and ligated to the 182 bp fragment containing the β-interferon 5′ flanking region. The ligation products are digested with BamHI and SmaI, and the 1742 bp SmaI-BamHI fragment, resulting from ligation of β-interferon sequences (position −173 with respect to the AUG translational initiation codon) to CMV IE sequences (−598 relative to the CMV IE CAP site), is gel purified. The 1742 bp SmaI-BamHI fragment is ligated to BamHI and SmaI digested plasmid pBSIISK⁺ (Stratagene Inc., La Jolla, Calif.) and electroporated into E. coli. Colonies containing inserts in pBSIISK⁺ are analyzed by restriction enzyme analysis to confirm the structure of the insert. One recombinant plasmid is identified and designated pBS-CB.

Oligo 7.1
(SEQ ID NO: 25)
5′ TGACATAGGA AAACTGAAAG G
Oligo 7.2
(SEQ ID NO: 26)
5′ TTTGGATCCG TTGACAACAC GAACAGTGTC G
Oligo 7.3
(SEQ ID NO: 27)
5′ TTTCCCGGGA CATTGATTAT TGACTAGTT
Oligo 7.4
(SEQ ID NO: 28)

5′ CGTGTCAAGG ACGGTGACTG C

The neomycin phosphocransferase (neo) gene is isolated from plasmid pBSneo for use as a selectable marker for the isolation of stably transfected human cells. The neo gene in plasmid pBSneo was obtained by BamHI and XhoI digestion of pMC1neo-polyA (Thomas, K. R. and Capecchi, M. R., Cell 51:503-512 (1987)). Plasmid pMC1neo-polyA was digested with BamHI and made blunt ended with the Klenow fragment of E. coli DNA polymerase I. The resulting DNA was digested with XhoI, and the blunt-ended BamHI-XhoI fragment was cloned into HincII and XhoI digested plasmid pBSIISK⁺. For isolation of the neo gene harbored on pBSneo, plasmid pBSneo is digested with XhoI and made blunt-ended by treatment with the Klenow fragment of E. coli DNA polymerase I. The resulting DNA is digested with HindIII and a 1165 bp fragment containing the neo expression unit is gel purified. The 1165 bp fragment is ligated to SmaI and HindIII digested plasmid pBS-CB and electroporated into E. coli. Colonies containing inserts in pBS-CE are analyzed by restriction enzyme analysis to confirm the orientation of the insert. One recombinant plasmid is identified and designated pBS-CBN.

Next, the dhfr expression unit is inserted at the ClaI site which is located at the 3′ end of the neo gene of pBS-CBN. The dhfr expression unit is obtained by EcoRI and SalI digestion of plasmid pF8CIS9080 (Eaton et al., Biochemistry 25:8343-8347 (1986)). The resultant 2 kb fragment is purified from the digest and made blunt with the Klenow fragment of E. coli DNA polymerase I. A ClaI linker (5′CCATCGATGG; NEB 1088, New England Biolabs, Beverly, Mass.) is ligated to the blunt-end dhfr fragment, the ligation products are digested with ClaI and purified. The ClaI dhfr containing fragment is ligated into ClaI digested plasmid pBS-CBN. An aliquot of the ligation reaction is electroporated into E. coli and colonies harboring inserts in a ClaI site of pBS-CBN are analyzed by restriction enzyme analysis to determine the site of insertion and the orientation of the insert. A plasmid with the dhfr expression unit at the 3′ end of the neo gene and with the same transcriptional orientation as that of the neo gene is identified and designated pBS-CBND.

Finally, the targeting construct is constructed by insertion of the 5′ targeting sequence (FIG. 16) in the unique SalI site located at the 3′ end of the dhfr expression unit in plasmid pBS-CBND. To obtain the 5′ targeting sequence, the plasmid pBS-H3/Bint.11-3 is digested with EcoRI and PvuII and the resultant 1.2 kb fragment is purified, ligated to EcoRI-SmaI digested plasmid pBSIISK⁺ (Stratagene Inc., La, Jolla, Calif.) and electroporated into E. coli. Colonies containing inserts in pBSIISK⁺ are analyzed by restriction enzyme analysis, and one plasmid containing the insert is retained and designated pBS-BI5. Plasmid pBS-BI5 is digested with SpeI and EcoRV and made blunt-ended with the Klenow fragment of DNA polymerase I. The resulting 1.2 kb fragment is ligated to SalI digested plasmid pBS-CBND, which has been made blunt-ended with the Klenow fragment of E. coli DNA polymerase I. An aliquot of the blunt-end ligation reaction is electroporated into E. coli and colonies harboring inserts in the SalI site of pBS-CBND are analyzed by restriction enzyme analysis to determine the orientation of the insert. A plasmid with the EcoRI site at the 3′ end of the dhfr expression unit is identified and designated pIFNβ-1.

Plasmid pIFNβ-1 is digested with BamHI for transfection into human cells. Transfection of primary, secondary, or immortalized human cells and isolation of homologously recombinant cells expressing β-interferon may be accomplished using the methods described in U.S. Ser. No. 08/243,391 and incorporated herein by reference. Homologously recombinant cells may be identified by PCR screening strategy as exemplified therein and in published methods available to one skilled in the art (see, for example, Kim, H-S and Smithies, O., Nucl. Acids Res. 16:8887-8903 (1988)). The identification of cells expressing β-interferon may also be accomplished using a variety of assays based on the structure or properties of β-interferon. For example, β-interferon may be identified by an in vitro reverse passive hemagglutlnation assay (Accurate Chemical Corp., Westbury, N.Y.), stimulation of superoxide anion production by mouse peritoneal macrophages (Colligan, J. E. et al. Current Protocols in Immunology, Wiley, New York, N.Y. (1994), or by using anti-β-interferon antibodies in an ELISA assay.

The isolation of cells containing amplified copies of the amplifiable marker gene and the activated β-interferon locus is performed as described in U.S. Ser. No. 07/985,586 incorporated herein by reference.

Equivalents

Those skilled in the art will recognize, or be able to ascertain using not more than routine experimentation, many equivalents to the specific embodiments of the invention described herein. Such equivalents are intended to be encompassed by the following claims.

1. A DNA construct capable of altering the expression of a gene encoding thrombopoietin when inserted by homologous recombination into chromosomal DNA of a cell, said construct comprising:
(a) a targeting sequence comprising DNA which hybridizes to genomic DNA within or upstream of the thrombopoietin gene;
(b) a regulatory sequence;
(c) an exon; and
(d) an unpaired splice-donor site.

2. The DNA construct of claim 1 wherein the regulatory sequence comprises a promoter.

3. The DNA construct of claim 2 further comprising a selectable marker gene.

4. The DNA construct of claim 2 further comprising an amplifiable marker gene.

5. The DNA construct of claim 1 further comprising a second targeting sequence comprising DNA which hybridizes to genomic DNA within or upstream of the thrombopoietin gene.

6. The DNA construct of claim 1 wherein the targeting sequence is selected from the group consisting of SEQ ID NO: 3, SEQ ID NO: 4 or fragments thereof or a sequence which hybridizes to a sequence selected from the group consisting of SEQ ID NO: 3, SEQ ID NO: 4 or fragments thereof.

7. The DNA construct of claim 6 wherein the targeting sequence is a fragment of SEQ ID NO: 3 and is at least about 20 base pairs.

8. The DNA construct of claim 6 wherein the targeting sequence is a fragment of SEQ ID NO: 4 and is at least about 20 base pairs.

9. The DNA construct of claim 8 wherein the targeting sequence is at least about 20 base pairs and is a sequence between about nucleotides −1815 to −145, 14 to 245, or 374 to 570 of FIG. 5 (SEQ ID NO: 4).

10. An isolated DNA molecule of at least about 20 base pairs selected from the group consisting of SEQ ID NO: 3, a fragment thereof, and a sequence which hybridizes to SEQ ID NO: 3.

11. An isolated DNA molecule of at least about 20 base pairs which is selected from the group consisting of a sequence between about nucleotides −1815 to −145, 14 to 245, or 374 to 570 of FIG. 5 (SEQ ID NO: 4), and a sequence which hybridizes to a sequence between about nucleotides −1815 to −145, 14 to 245, or 374 to 570 of FIG. 5 (SEQ ID NO: 4).

12. A method of producing a homologously recombinant cell wherein the expression of the thrombopoietin gene is altered, comprising the steps of:
(a) transfecting a cell containing the thrombopoietin gene with the DNA construct of one of claims 1-9; and
(b) maintaining the transfected cell under conditions appropriate for homologous recombination.

13. A homologously recombinant cell produced by the method of claim 12.

14. A homologously recombinant cell which expresses thrombopoietin comprising an exogenous regulatory region, an exogenous exon, and an exogenous unpaired splice-donor site operatively linked to an endogenous splice acceptor site of the thrombopoietin gene.

15. The homologously recombinant cell of claim 14 wherein the exogenous regulatory region, the exogenous exon, and the exogenous unpaired splice-donor site are operatively linked to the endogenous splice acceptor site of the second or third exon of the thrombopoietin gene.

16. A method for producing thrombopoietin comprising the steps of maintaining the homologously recombinant cell of claim 14 or 15 under conditions appropriate for the production of thrombopoietin.

17. A method for producing thrombopoietin wherein the expression of the thrombopoietin gene is altered, comprising the steps of:
(a) transfecting a cell containing the thrombopoietin gene with the DNA construct of one of claims 1-9; and
(b) maintaining the transfected cell under conditions appropriate for homologous recombination; and
(c) maintaining the homologously recombinant cell produced in step (b) under conditions appropriate for the production of thrombopoietin.

18. A thrombopoietin produced by the method of claim 17.

19. A pharmaceutical composition comprising the thrombopoietin of claim 18.

20. A method of providing thrombopoietin to a mammal in need thereof comprising administering homologously recombinant cells of claim 14 or 15 in sufficient number to produce a therapeutically effective amount of thrombopoietin in the mammal.

21. A DNA construct capable of altering the expression of a gene encoding DNase I when inserted by homologous recombination into chromosomal DNA of a cell, said construct comprising:
(a) a targeting sequence comprising DNA which hybridizes to genomic DNA within or upstream of the DNase I gene;
(b) a regulatory sequence;
(c) an exon; and
(d) an unpaired splice-donor site.

22. The DNA construct of claim 21 wherein the regulatory sequence comprises a promoter.

23. The DNA construct of claim 22 further comprising a selectable marker gene.

24. The DNA construct of claim 22 further comprising an amplifiable marker gene.

25. The DNA construct of claim 21 further comprising a second targeting sequence comprising DNA which hybridizes to genomic DNA within or upstream of the DNase I gene.

26. The DNA construct of claim 21 wherein the targeting sequence is selected from the group consisting of SEQ ID NO: 17, SEQ ID NO: 18 or fragments thereof or a sequence which hybridizes to a sequence selected from the group consisting of SEQ ID NO: 17, SEQ ID NO: 18 or fragments thereof.

27. The DNA construct of claim 26 wherein the targeting sequence is a fragment of SEQ ID NO: 17 and is at least about 20 base pairs.

28. The DNA construct of claim 26 wherein the targeting sequence is a fragment of SEQ ID NO: 18 and is at least about 20 base pairs.

29. The DNA construct of claim 28 wherein the targeting sequence is at least about 20 base pairs and is a sequence between about nucleotides −328 to −2 of FIG. 11 (SEQ ID NO: 18).

30. An isolated DNA molecule of at least about 20 base pairs selected from the group consisting of SEQ ID NO: 17, a fragment thereof, and a sequence which hybridizes to SEQ ID NO: 17.

31. An isolated DNA molecule of at least about 20 base pairs which is selected from the group consisting of a sequence between about nucleotides −328 to −2 of FIG. 11 (SEQ ID NO: 18) and a sequence which hybridizes to a sequence between about nucleotides −328 to −2 of FIG. 11 (SEQ ID NO: 18).

32. A method of producing a homologously recombinant cell wherein the expression of the DNase I gene is altered, comprising the steps of:
(a) transfecting a cell containing the DNase I gene with the DNA construct of one of claims 21-29; and
(b) maintaining the transfected cell under conditions appropriate for homologous recombination.

33. A homologously recombinant cell produced by the method of claim 32.

34. A homologously recombinant cell which expresses DNase I comprising an exogenous regulatory region, an exogenous exon, and an exogenous unpaired splice-donor site operatively linked to an endogenous splice acceptor site of the DNase I gene.

35. The homologously recombinant cell of claim 34 wherein the exogenous regulatory region, the exogenous exon, and the exogenous unpaired splice-donor site are operatively linked to the endogenous splice acceptor site of the second exon of the DNase I gene.

36. A method for producing DNase I comprising the steps of maintaining the homologously recombinant cell of claim 34 or 35 under conditions appropriate for the production of DNase I.

37. A method for producing DNase I wherein the expression of the DNase I gene is altered, comprising the steps of:
(a) transfecting a cell containing the DNase I gene with the DNA construct of one of claims 21-29; and
(b) maintaining the transfected cell under conditions appropriate for homologous recombination; and
(c) maintaining the homologously recombinant cell produced in step (b) under conditions appropriate for the production of DNase I.

38. A DNase I produced by the method of claim 37.

39. A pharmaceutical composition comprising the DNase I of claim 38.

40. A method of providing DNase I to a mammal in need thereof comprising administering homologously recombinant cells of claim 34 or 35 in sufficient number to produce a therapeutically effective amount of DNase I in the mammal.

41. A DNA construct capable of altering the expression of a gene encoding β-interferon when inserted by homologous recombination into chromosomal DNA of a cell, said construct comprising:
(a) a targeting sequence comprising DNA which hybridizes to genomic DNA within or upstream of the β-interferon gene;
(b) a regulatory sequence;
(c) an exon;
(d) a splice-donor site;
(e) an intron; and
(f) a splice-acceptor site

42. The DNA construct of claim 41 wherein the regulatory sequence comprises a promoter.

43. The DNA construct of claim 42 further comprising a selectable marker gene.

44. The DNA construct of claim 42 further comprising an amplifiable marker gene.

45. The DNA construct of claim 41 further comprising a second targeting sequence comprising DNA which hybridizes to genomic DNA within or upstream of the β-interferon gene.

46. The DNA construct of claim 41 wherein the targeting sequence is selected from the group consisting of SEQ ID NO: 23, SEQ ID NO: 24 or fragments thereof or a sequence which hybridizes to a sequence selected from the group consisting of SEQ ID NO: 23, SEQ ID NO: 24 or fragments thereof.

47. The DNA construct of claim 46 wherein the targeting sequence is a fragment of SEQ ID NO: 23 and is at least about 20 base pairs.

48. The DNA construct of claim 46 wherein the targeting sequence is a fragment of SEQ ID NO: 24 and is at least about 20 base pairs.

49. An isolated DNA molecule of at least about 20 base pairs selected from the group consisting of SEQ ID NO: 23, a fragment thereof, and a sequence which hybridizes to SEQ ID NO: 23.

50. A method of producing a homologously recombinant cell wherein the expression of the β-interferon gene is altered, comprising the steps of:
(a) transfecting a cell containing the β-interferon gene with the DNA construct of one of claims 41-48; and
(b) maintaining the transfected cell under conditions appropriate for homologous recombination.

51. A homologously recombinant cell produced by the method of claim 50.

52. A homologously recombinant cell which expresses β-interferon comprising an exogenous regulatory region, an exogenous exon, an exogenous splice-donor site, and exogenous intron, and an exogenous splice acceptor site operatively linked to the β-interferon gene.

53. A method for producing β-interferon comprising the steps of maintaining the homologously recombinant cell of claim 52 under conditions appropriate for the production of β-interferon.

54. A method for producing β-interferon wherein the expression of the β-interferon gene is altered, comprising the steps of:
(a) transfecting a cell containing the β-interferon gene with the DNA construct of one of claims 41-48; and
(b) maintaining the transfected cell under conditions appropriate for homologous recombination; and
(c) maintaining the homologously recombinant cell produced in step (b) under conditions appropriate for the production of β-interferon.

55. A β-interferon produced by the method of claim 54.

56. A pharmaceutical composition comprising the β-interferon of claim 55.

57. A method of providing β-interferon to a mammal in need thereof comprising administering homologously recombinant cells of claim 52 in sufficient number to produce a therapeutically effective amount of β-interferon in the mammal.
